(19) United States
(12) Patent Application Publication
(10) Pub. No.: US 2006/0270863 A1
(43) Pub. Date: Nov. 30, 2006

(54) CONVERSION OF AMORPHA-4,11-DIENE TO ARTEMISININ AND ARTEMISININ PRECURSORS

(75) Inventors: Keith Kinkead Reiling, Oakland, CA (US); Neil Stephen Renninger, Oakland, CA (US); Derek James McPhee, Richmond, CA (US); Karl Joseph Fisher, Petaluma, CA (US); Denise Ann Ockey, Richmond, CA (US)

Correspondence Address: WILSON SONSINI GOODRICH & ROSATI, 650 PAGE MILL ROAD, PALO ALTO, CA, 94304-1050, US

(73) Assignee: Amyris Biotechnologies, Emeryville, CA (US)

(21) Appl. No.: 11/419,975

(22) Filed: May 23, 2006

Related U.S. Application Data
(60) Provisional application No. 60/685,713, filed on May 27, 2005.
(60) Provisional application No. 60/775,517, filed on Feb. 21, 2006.

(51) Int. Cl. C07D 493/14; C07C 61/12; C07D 301/14; C07C 61/28; C07D 487/22

(52) U.S. Cl. 549/361; 562/501; 549/525

(57) ABSTRACT

The present invention relates to methods for the conversion of amorpha-4,11-diene to artemisinin and various artemisinin precursors.

### CROSS-REFERENCES TO RELATED APPLICATION

The present application claims priority to U.S. Provisional Patent Application No. 60/685,713, filed May 27, 2006; and U.S. Provisional Patent Application No. 60/775,517, filed Feb. 21, 2006, each of which is incorporated herein by reference in their entirety for all purposes.

### BACKGROUND OF THE INVENTION

Approximately 270 million people are infected with malaria, making it one of the world's major infectious diseases. Developing new anti-malarial drugs, and alternative methods of producing anti-malarial drugs, is therefore an important world health objective.

One of these anti-malarial drugs is artemisinin (compound 4 in Table 1). Artemisinin is a component of the traditional Chinese medicinal herb Artemisia annua, which has been utilized for controlling symptoms of fever in China for over 1000 years. In the scientific literature, artemisinin is also sometimes referred to by its Chinese name, Qinghaosu. Recent strides have been made in understanding the properties and structure of this molecule. The compound was first isolated in 1972. Its anti-malarial activity was discovered in 1979 (Chinese Med. J., 92: 811 (1979)). The total synthesis of the molecule was accomplished in 1983 (Schmid, G., Hofheinz, W., J. Am. Chem. Soc., 105: 624 (1983)).

Production of artemisinin 4 can be accomplished through several routes. One method involves extracting artemisinin from Artemisia annua. A drawback of this method is the low and inconsistent yields (0.01-0.8%) of artemisinin from the plant (Wallart, et al., Planta Med 66: 57-62 (2000); Abdin, et al., Planta Med 69: 289-299 (2003)). An alternate production procedure involves extracting an artemisinin precursor, artemisinic acid (compound 2 in Table 1), from Artemisia annua and then synthetically converting this molecule into artemisinin. Because 2 can be present in Artemisia annua at levels approximately 10 times higher than 4, the conversion of the former to the latter has received a great deal of attention. However, the yields of artemisinic acid from Artemisia annua are variable and despite the quick growth of Artemisia annua, it is currently estimated that the world's supply of the plant would meet less than 10% of the world's demand for artemisinic acid and artemisinin. Therefore, artemisinic acid is generally considered to be inaccessible (Haynes et al., Chem. Bio. Chem., 6: 659-667 (2005)) and, a need for an economical and scalable method of producing artemisinin remains.

A synthetic route for the conversion of artemisinic acid to artemisinin via dihydroartemisinic acid (DHAA, compound 3 in Table 1) has been described in U.S. Pat. No. 4,992,561 to Roth et al. Therefore, a reliable and cost-effective source of DHAA 3 would provide an important step towards a sustainable method of producing the anti-malaria compound artemisin 4. The current invention addresses this and other needs.

One possible route to synthesize DHAA 3 starts with the sesquiterpene hydrocarbon amorpha-4,11-diene (compound 1 in Table 1), an accessible starting material. A method of preparing amorpha-4,11-diene via recombinant technology has been described in U.S. Patent Application No. 20040005678 to Keasling et al. The process of large-scale production of amorpha-4,11-diene is further described in U.S. Patent Application No. 20040005678.

The transformation of amorpha-4,11-diene 1 to DHAA 3 requires the selective functionalization of the exocyclic alkene (C11-C12) in the presence of the endocyclic alkene (C4-C5).

While reliable and robust methods for the selective epoxidation of functionalized alkenes are available, for instance the well known Sharpless expoxidation of allylic alcohols, selective modifications of unfunctionalized systems are generally difficult to achieve. For example, Thomas and Bessiere (Nat. Prod. Rep., 291 (1989) and references cited therein) teach that in the case of (+) limonene, which contains both an endocyclic and an exocyclic double bond, the endocyclic double bond is epoxidized preferentially, even though the exocyclic bond is sterically more accessible to potential oxidizing reagents. This fact is attributed to the greater nucleophilicity of the endocyclic double bond (Figure I).

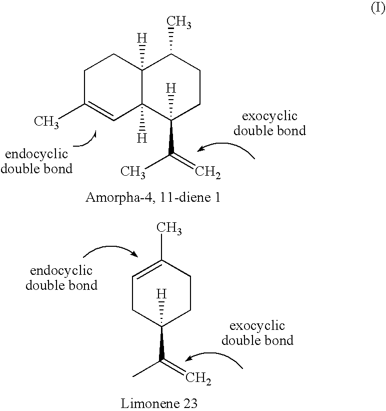

Epoxidations of exocyclic double bonds in the presence of endocyclic double bonds using common epoxidation reagents usually afford mixtures of mono- and diepoxides in which the endocyclic monoepoxide predominates. For example, the epoxidation of (+)-limonene (compound 23 in Figure I) with peracids affords a mixture of epoxides, which contains only 10% of the exocyclic monoepoxide. Various other methods lead to only modest variations in this proportion.

Attempts have been made to direct the epoxidation of limonene towards the less encumbered exocyclic alkene by using sterically demanding oxidants, such as metallosalenes, metalloporphyrins or other large metallic complexes in the presence of an oxygen donor. But even when using the most sterically hindered porphyrin reported, the selectivity was found to be poor (50-60%) (Suslick et al., J. Am Chem. Soc., 118:5708-5711 (1996)). Recently, higher selectivity towards the exocyclic monoepoxide has been achieved by biotransformation, using a unique strain of Xanthobacter (van der Werf et al., J. Biotechnol. 84:133 (2000)) and by chemical oxidation using a polyoxovanadometalate catalyst and hydrogen peroxide (Mizuno et al, Angew. Chem. Int. Ed., 44: 5136 (2005)). However, the synthesis of the described catalyst is cumbersome. Therefore, the current method of choice for obtaining the exocyclic epoxide of (+)-limonene selectively via chemical synthesis requires protection of the endocyclic alkene prior to epoxidation of the exocyclic alkene and later regeneration of the endocyclic alkene (Almeida et al., Synth. Commun., 35: 1285 (2005)).

The prior art teaches that even the simultaneous use of a bulky catalyst and increased steric hindrance around the endocyclic double bond may not be sufficient to overcome the higher reactivity of this alkene. Maraval et al. (J. Catalysis, 206: 349 (2002)) teach that the epoxidation of the monoterpene derived substrate 5-vinyl-2-norbornene (compound 24) using a variety of different metalloporphyrin catalysts, leads to the endocyclic mono-exo-epoxide (compound 25) despite the fact that the bridgehead carbon provides steric hindrance at the endocyclic alkene position while the exocyclic alkene is sterically unhindered.

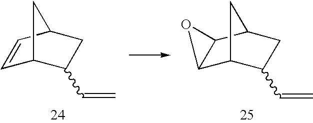

The selective epoxidation of sesquiterpene substrates is similarly challenging. For example, in the case of (+) valencene (compound 26, below), epoxidation with peracids gives a 3.5:1 mixture of the endocyclic monoepoxide and the diepoxide (Shaffer et al., J. Org. Chem., 47: 2181 (1975)).

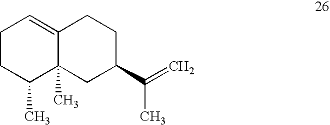

While the regioselective synthesis of the endocyclic monoepoxide of (+) valencene has been reported (Ali et al., Tetrahedron Lett, 47: 8769 (2002)), the exocyclic monoepoxide has not yet been synthesized selectively. The molecule was characterized as a minor component of the complex mixture of oxidation products resulting from m-chloroperbenzoic acid oxidation of (+) valencene or bioconversion of the same substrate with the ascomycete Chaetomium globosum (Berger et al., Appl. Microbiol. Biotechnol., 67: 477 (2005)). The molecule was also isolated in very small amounts from Alaskan yellow cedar and was found to possess potent insecticidal properties (Dolan et al., US Pat. Appl. 2005/0187289).

Thus, a synthetic method for the selective oxidation of an exocyclic double bond in a substrate molecule comprising one or more endocyclic double bonds would represent a significant advancement in the art. The present invention addresses this and other needs.

### BRIEF SUMMARY OF THE INVENTION

In one aspect, the present invention provides a method of regioselectively epoxidizing an exocyclic alkene over an endocyclic alkene, such method comprising contacting a substrate and an epoxidation oxidant and a member selected from a metalloporphyrin and a metallosalen.

In another aspect the invention provides a method of regioselectively dihydroxylating an exocyclic alkene over an endocyclic alkene, such method comprising contacting a substrate and a dihydroxylation reagent, which comprises a transition metal based oxidant or catalyst.

The invention further provides methods of preparing dihydroartemisinic acid from amorpha-4,11-diene.

In another aspect the invention provides methods of preparing artemisinin and artemisinin analogs. The methods of the invention can also be utilized to synthesize the compounds in large scale quantities.

### DETAILED DESCRIPTION OF THE INVENTION

I. Abbreviations and Definitions

DHAA=DiHydroArtemisinic Acid.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multi-valent radicals, having the number of carbon atoms designated (i.e. C₁-C₁₀ means one to ten carbons). Examples of saturated hydrocarbon radicals include groups such as methyl, ethyl, n-propyl, isopropyl, n-butyl, t-butyl, isobutyl, sec-butyl, cyclohexyl, (cyclohexyl)ethyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below as “heteroalkyl,” “cycloalkyl” and “alkylene.” The term “alkylene” by itself or as part of another substituent means a divalent radical derived from an alkane, as exemplified by —CH₂CH₂CH₂CH₂—. Typically, an alkyl group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” refer to those groups having an alkyl group attached to the remainder of the molecule through an oxygen, nitrogen or sulfur atom, respectively. Similarly, the term “dialkylamino” is used in a conventional sense to refer to —NR′R″ wherein the R groups can be the same or different alkyl groups.

The term “acyl” or “alkanoyl” by itself or in combination with another term, means, unless otherwise stated, a stable straight or branched chain, or cyclic hydrocarbon radical, or combinations thereof, consisting of the stated number of carbon atoms and an acyl radical on at least one terminus of the alkane radical.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Also included in the term “heteroalkyl” are those radicals described in more detail below as “heteroalkylene” and “heterocycloalkyl.” The term “heteroalkylene” by itself or as part of another substituent means a divalent radical derived from heteroalkyl, as exemplified by —CH₂—CH₂—S—CH₂CH₂— and —CH₂—S—CH₂—CH₂—NH—CH₂—. For heteroalkylene groups, heteroatoms can also occupy either or both of the chain termini. Still further, for alkylene and heteroalkylene linking groups, no orientation of the linking group is implied.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include cyclopentyl, cyclohexyl, 1-cyclohexenyl, 3-cyclohexenyl, cycloheptyl, and the like. Examples of heterocycloalkyl include 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “fluoroalkyl,” are meant to include monofluoroalkyl and polyfluoroalkyl.

The term “aryl,” employed alone or in combination with other terms (e.g., aryloxy, arylthioxy, arylalkyl) means, unless otherwise stated, an aromatic substituent which can be a single ring or multiple rings (up to three rings), which are fused together or linked covalently. “Heteroaryl” are those aryl groups having at least one heteroatom ring member. Typically, the rings each contain from zero to four heteroatoms selected from N, O, and S, wherein the nitrogen and sulfur atoms are optionally oxidized, and the nitrogen atom(s) are optionally quaternized. The “heteroaryl” groups can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl ring systems are selected from the group of acceptable substituents described below. The term “arylalkyl” is meant to include those radicals in which an aryl group is attached to an alkyl group (e.g., benzyl, phenethyl, pyridylmethyl and the like) or a heteroalkyl group (e.g., phenoxymethyl, 2-pyridyloxymethyl, 3-(1-naphthyloxy)propyl, and the like).

Each of the above terms (e.g., “alkyl,” “heteroalkyl” and “aryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be a variety of groups selected from, for example: —OR′, ═O, ═NR′, ═N—OR′, —NR′R″, —SR′, -halogen, —SiR′R″ R′″, —OC(O)R′, —C(O)R′, —CO₂R′, CONR′R″, —OC(O)NR′R″, —NR″C(O)R′, —NR′—C(O)NR″R′″, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, —CN and —NO₂ in a number ranging from zero to (2N+1), where N is the total number of carbon atoms in such radical. R′, R″ and R′″ each independently refer to hydrogen, unsubstituted (C₁-C₈)alkyl and heteroalkyl, unsubstituted aryl, aryl substituted with 1-3 halogens, unsubstituted alkyl, alkoxy or thioalkoxy groups, or aryl-(C₁-C₄)alkyl groups. When R′ and R″ are attached to the same nitrogen atom, they can be combined with the nitrogen atom to form a 5-, 6-, or 7-membered ring. For example, —NR′R″ is meant to include 1-pyrrolidinyl and 4-morpholinyl. From the above discussion of substituents, one of skill in the art will understand that the term “alkyl” is meant to include groups such as haloalkyl (e.g., —CF₃ and —CH₂CF₃) and acyl (e.g., —C(O)CH₃, —C(O)CF₃, —C(O)CH₂OCH₃, and the like).

Similarly, substituents for the aryl groups are varied and are selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, (C₁-C₈)alkyl and heteroalkyl, unsubstituted aryl, (unsubstituted aryl)-(C₁-C₄)alkyl, (unsubstituted aryl)oxy-(C₁-C₄)alkyl and perfluoro(C₁-C₄)alkyl.

Two of the substituents on adjacent atoms of the aryl ring may optionally be replaced with a substituent of the formula -T-C(O)—(CH₂)_{q}—U—, wherein T and U are independently —NH—, —O—, —CH₂— or a single bond, and the subscript q is an integer of from 0 to 2. Alternatively, two of the substituents on adjacent atoms of the aryl ring may optionally be replaced with a substituent of the formula -A-(CH₂)ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl ring may optionally be replaced with a substituent of the formula —(CH₂)ₙ—X—(CH₂)ₜ—, where s and t are independently integers of from 0 to 3, and X is —O—, —NR′—, —S—, —S(O)—, —S(O)₂—, or —S(O)₂NR′—. The substituent R⁹ in —NR′— and —S(O)₂NR′— is selected from hydrogen or unsubstituted (C₁-C₆)alkyl.

As used herein, the term “heteroatom” is meant to include, for example, oxygen (O), nitrogen (N), sulfur (S) and silicon (Si).

Certain compounds of the present invention possess asymmetric carbon atoms (optical centers) or double bonds; the racemates, diastereomers, geometric isomers and individual isomers are all encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

As used herein, the term “leaving group” refers to a portion of a substrate that is cleaved from the substrate in a reaction. The leaving group is an atom (or a group of atoms) that is displaced as a stable species taking with it the bonding electrons. Typically the leaving group is an anion (e.g., Cl⁻) or a neutral molecule (e.g., H₂O). Exemplary leaving groups include a halogen, OC(O)R⁹, OP(O)R⁹R¹⁰, OS(O)R⁹, and OSO₂R⁹. R⁹ and R¹⁰ are members independently selected from substituted or unsubstituted alkyl, substituted or unsubstituted aryl, substituted or unsubstituted heteroaryl and substituted or unsubstituted heterocycloalkyl. Useful leaving groups include, but are not limited to, other halides, sulfonic esters, oxonium ions, alkyl perchlorates, sulfonates, e.g., arylsulfonates, ammonioalkanesulfonate esters, and alkylfluorosulfonates, phosphates, carboxylic acid esters, carbonates, ethers, and fluorinated compounds (e.g., triflates, nonaflates, tresylates), S R⁹, (R⁹)₃P⁺, (R⁹)₂S⁺, P(O)N(R⁹)₂(R⁹)₂, P(O)XR⁹X′R⁹ in which each R⁹ is independently selected from the members provided in this paragraph and X and X′ are S or O. The choice of these and other leaving groups appropriate for a particular set of reaction conditions is within the abilities of those of skill in the art (see, for example, March J, ADVANCED ORGANIC CHEMISTRY, 2nd Edition, John Wiley and Sons, 1992; Sandler S R, Karo W, ORGANIC FUNCTIONAL GROUP PREPARATIONS, 2nd Edition, Academic Press, Inc., 1983; and Wade L G, COMPENDIUM OF ORGANIC SYNTHETIC METHODS, John Wiley and Sons, 1980).

“Protecting group,” as used herein refers to a portion of a substrate that is substantially stable under a particular reaction condition, but which is cleaved from the substrate under a different reaction condition. A protecting group can also be selected such that it participates in the direct oxidation of the aromatic ring component of the compounds of the invention. For examples of useful protecting groups, see, for example, Greene et al., PROTECTIVE GROUPS IN ORGANIC SYNTHESIS, 3rd ed., John Wiley & Sons, New York, 1999.

The term “chiral transition metal catalyst” refers to a catalyst comprising a transition metal, including but not limited to Ni, Pd, Pt, Ru, Rh, Re or mixtures of these elements. Chiral transition metal catalysts also comprise one or more chiral ligands known in the art to confer enantioselectivity to the reactions in which they are used. These chiral transition metal catalysts may be homogeneous (i.e. soluble in the reaction medium) or heterogeneous (i.e. insoluble in the reaction medium). Chiral transition metal catalysts may also further comprise a solid support conferring insolubility such as, but not limited to, carbon, silica, alumina, an inorganic salt or a polymeric substance.

The term “metallosalen” refers to a catalyst comprising a metal, frequently Mn, but also Ti, V, Ru, Co, Cr, etc and an optically active N,N′-ethylenebis(salicylideneaminato) ligand, resulting from the reaction of a salicylaldehyde derivative, a diamine and a metal ion. The term “metalloporphyrin” refers to a natural or synthetic substance comprised of a substituted or unsubstituted framework formed of four pyrrole rings joined together by methylene bridges and surrounding a metal ion, and usually including, depending on the oxidation state of the metal ion, additional ligands and counterions. Important natural metalloporphyrins include chlorophyll and heme in blood, which participate in natural oxidation processes. Numerous synthetic metalloporphyrins are known that mimic these natural ones by acting as oxidation catalysts in the presence of suitable oxygen donors.

The term “regioselective” refers to the tendency of a chemical reaction to proceed so that a product resulting from reaction at one site within the substrate is formed over the product resulting from reaction at other sites within the substrate. For example an epoxidation reaction is called regioselective if epoxidation occurs predominantly at one alkene bond over another alkene bond within the same substrate.

II. Introduction

In one aspect the present invention provides a method for the regioselective epoxidation and regioselective dihydroxylation of an exocyclic alkene in a substrate molecule comprising one or more endocyclic alkenes. The invention further provides methods of converting amorpha-4,11-diene (compound 1 in Table 1) to dihydroartemisinic acid (compound 3 in Table 1). In another aspect the invention provides methods for the conversion of amorpha-4,11-diene 1 to artemisinin (compound 4 in Table 1). The methods of the invention can also be utilized to synthesize the compounds in large-scale quantities. Table 1 below provides the names and structures of the relevant compounds in the invention.

TABLE 1

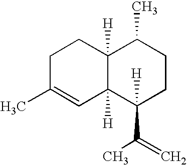

Compound 1: Amorpha-4,11-diene

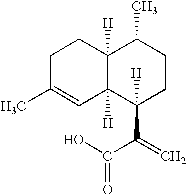

Compound 2: Artemisinic Acid

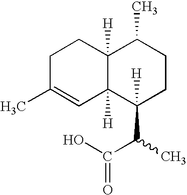

Compound 3: DiHydroArtemisinic Acid (DHAA)

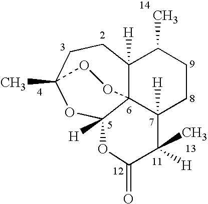

Compound 4: Artemisinin

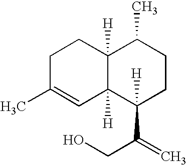

Compound 5: Amorph-4-ene-12-ol

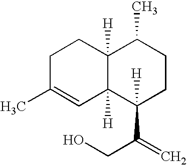

Compound 6: Amorpha-4,11-diene-13-ol

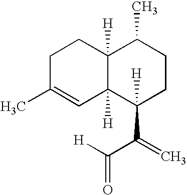

Compound 7: Amorph-4-ene-12-al

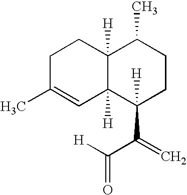

Compound 8: Amorpha-4,11-diene-13-al

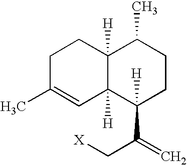

Compound 9

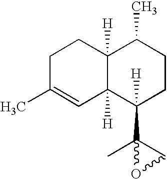

Compound 10

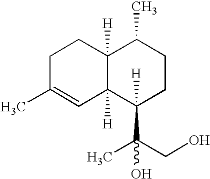

Compound 11

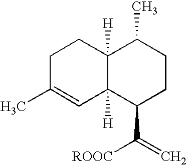

Compound 12

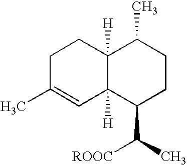

Compound 13

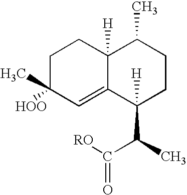

Compound 14

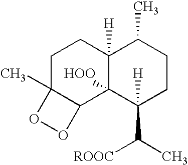

Compound 15

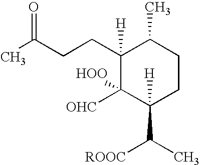

Compound 16

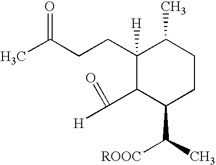

Compound 17

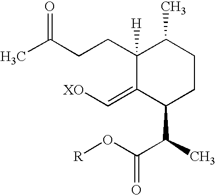

Compound 18

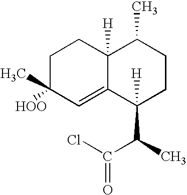

Compound 19

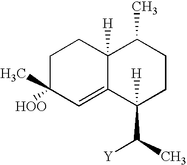

Compound 20

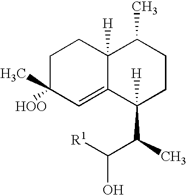

Compound 21

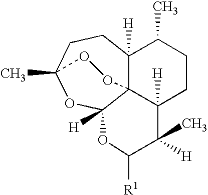

Compound 22

III. Regioselective Oxidation of an Exocyclic Alkene over an Endocyclic Alkene
III. a.) Epoxidation

In one aspect the current invention provides a method of regioselectively epoxidizing an exocyclic alkene over an endocyclic alkene, said method comprising contacting a substrate and an epoxidation oxidant and a member selected from a metalloporphyrin and a metallosalen.

In an exemplary embodiment wherein the substrate comprises one exocyclic double bond and one endocyclic double bond, the ratio (r) of the exocyclic epoxide (Ex) to the endocyclic epoxide (En) and the diepoxide (Di) in the final reaction mixture [r=Ex/(En+Di)] is about 50% to about 100%. In another exemplary embodiment the ratio is about 55% to 100%. In another exemplary embodiment the ratio is about 60% to about 100%. In another exemplary embodiment the ratio is about 65% to 100%. In another exemplary embodiment the ratio is about 70% to 100%. In another exemplary embodiment the ratio is about 75% to 100%. In another exemplary embodiment the ratio is about 80% to 100%. In another exemplary embodiment the ratio is about 85% to 100%. In another exemplary embodiment the ratio is about 90% to 100%. In another exemplary embodiment the ratio is about 95% to 100%.

In an exemplary embodiment the metal in the metalloporphyrin or the metallosalen is a transition metal. In another exemplary embodiment said transition metal is a member selected from chromium, manganese, iron, cobalt, nickel, copper, zinc, ruthenium, and palladium.

In another exemplary embodiment, the porphyrin portion in the metalloporphyrin is a member selected from TPP, TTMPP and TTP.

In another exemplary embodiment, the epoxidation oxidant is a member selected from oxygen, a peroxide, a peracid, a hypochlorite, a peroxydisulfate (S₂O₈²⁻), a dioxyrane, iodosylbenzene (PhIO), and combinations thereof. In an exemplary embodiment the peroxide is a member selected from hydrogen peroxide and t-BuOOH. In yet another exemplary embodiment the peracid is meta-chloroperbenzoic acid (mCPBA). In another exemplary embodiment, the peroxidisulfate is a member selected from sodium peroxidisulfate (Na₂S₂O₈), potassium peroxidisulfate (K₂S₂O₈) and ammonium peroxidisulfate, (NH₄)₂S₂O₈.

In another exemplary embodiment the oxidant is used in a stoichiometric excess. In an exemplary embodiment the oxidant is used in a stoichiometric excess of about 1.1 to about 10 equivalents. In a preferred embodiment the oxidant is used in a stoichiometric excess of about 4 to 6 equivalents.

In an exemplary embodiment the substrate of the epoxidation reaction is a member selected from a naturally occurring compound and a synthetic substrate. In a preferred embodiment the substrate is a cyclic, unsaturated hydrocarbon. In an exemplary embodiment, the substrate is a member selected from a monoterpene, a sesquiterpene, a diterpene, and a triterpene.

In a further exemplary embodiment, the sesquiterpene substrate is a member selected from an amorphane, a valencane, a cadinane, an eremophilane, a guaiane, a germacrane and a eudesmane. It shall be apparent to one skilled in the art that sesquiterpenes having other carbon skeletons can also be used in the methods of the invention.

In an exemplary embodiment the sesquiterpene is amorpha-4,11-diene 1.

In an exemplary embodiment, treatment of amorpha-4,11-diene 1 with a catalytic amount of the metalloporphyrin Mn(2,6-Cl₂TPP)Cl and a stoichiometric excess (5 equivalents) of hydrogen peroxide as the oxygen source results in the preferential formation of the corresponding exocyclic monoepoxide 10. In an exemplary embodiment compound 10 is the sole detectable product of the epoxidation reaction (Example 3.2.).

In another exemplary embodiment treatment of the sesquiterpene (+) valencene (compound 26) with a catalytic amount of the metalloporphyrin Mn(2,6-Cl₂TPP)Cl and a stoichiometric excess (5 equivalents) of hydrogen peroxide as the oxygen source results in the preferential formation of the corresponding exocyclic monoepoxide 31. In an exemplary embodiment compound 31 is the sole detectable product of the epoxidation reaction (Example 8).

III. b.) Dihydroxylation

In a second aspect the invention provides a method of regioselectively dihydroxylating an exocyclic alkene over an endocyclic alkene, said method comprising contacting a substrate with a dihydroxylation reagent, which comprises a transition metal based oxidant or catalyst.

In an exemplary embodiment wherein the substrate comprises one exocyclic double bond and one endocyclic double bond, the ratio (r1) of the exocyclic diol (Ex1) to the endocyclic diol (En1) and the product wherein both double bonds are oxidized to a diol (Di1) in the final reaction mixture [r1=Ex1/(En1+Di1)] is about 50% to about 100%. In another exemplary embodiment the ratio is about 55% to about 100%. In another exemplary embodiment the ratio is about 60% to 100%. In another exemplary embodiment the ratio is about 65% to 100%. In another exemplary embodiment the ratio is about 70% to 100%. In another exemplary embodiment the ratio is about 75% to 100%. In another exemplary embodiment the ratio is about 80% to 100%. In another exemplary embodiment the ratio is about 85% to 100%. In another exemplary embodiment the ratio is about 90% to 100%. In another exemplary embodiment the ratio is about 95% to 100%.

In an exemplary embodiment, the oxidant in the dihydroxylation reagent is a member selected from osmium tetraoxide (OsO₄) and ruthenium tetraoxide (RuO₄).

In another exemplary embodiment the dihydroxylation reagent further comprises a co-oxidant for the regeneration of the primary oxidant. In an exemplary embodiment the co-oxidant is a member selected from a peroxide, a peracid, a tertiary amine N-oxide, K₃Fe(CN)₆, a chlorite, I₂, a selenoxide and a peroxysulfate (S₂O₈²⁻). In a preferred embodiment the tertiary amine N-oxide is N-methylmorpholine-N-oxide (NMO).

In an exemplary embodiment the substrate of the dihydroxylation reaction is a member selected from a naturally occurring compound and a synthetic compound. In a preferred embodiment the substrate is a cyclic, unsaturated hydrocarbon. In an exemplary embodiment the substrate for the dihydroxylation reaction is a member selected from a monoterpene, a sesquiterpene, a diterpene and a triterpene.

In another exemplary embodiment, the sesquiterpene is a member selected from an amorphane, a valencane, a cadinane, an eremophilane, a guaiane, a germacrane and a eudesmane.

In another exemplary embodiment the sesquiterpene is amorpha-4,11-diene 1.

It is known to those skilled in the art that higher substituted olefins are typically oxidized faster than lower substituted olefins (Sharpless, K. B. and Anderson, P. G. J. Am Chem. Soc. 1993, 115, 7047-7048). It shall also be evident that the selectivity of the oxidation reaction decreases when less substituted olefins are placed in competition with terminal olefins. In these examples a mixture of products are observed (Sharpless, K. B. and Gerard, D. X. J. Am. Chem. Soc. 1992, 114, 7570-7571). Examples of dihydroxylation reagents may be found in March, loc. cit., pp. 822-825 or Larock, loc. cit. pp. 996-1001 and references therein.

In an exemplary embodiment, the current invention provides a method of regioselectively dihydroxylating an exocyclic alkene in the presence of endocyclic alkenes while preventing overoxidation of the resulting 1,2-diol and preventing the oxidative cleavage of the oxidized bond. In an exemplary embodiment the invention provides a method of regioselectively preparing dihydroxy derivatives of sesquiterpenes. In another exemplary embodiment, the oxidation of (+) valencene 26 with a catalytic amount of osmium tetroxide and N-methylmorpholine-N-oxide (NMO) results in the preferential formation of the exocyclic diol 32. In an exemplary embodiment the exocyclic diol of valencene 32 is the sole detectable oxidation product (Example 9).

In another exemplary embodiment, the oxidation of amorpha-4,11-diene 1 with a catalytic amount of osmium tetraoxide and N-methylmorpholine-N-oxide (NMO) results in the preferential formation of the exocyclic diol 11. In an exemplary embodiment the exocyclic diol 11 is the sole detectable oxidation product.

IV. Synthesis of DHAA 3 from Amorpha-4,11-diene 1

In one aspect, the invention provides a method of preparing DHAA, 3 from amorpha-4,11-diene, 1. The transformation of 1 to 3 can be accomplished through the reduction of the C₁₁-C₁₂ double bond in 1 and the introduction of a carboxylic acid functionality at C₁₂. The atom numbering for the compounds of the invention is consistent with the numbering scheme for 4 in Table 1. It shall be recognized by one skilled in the art that due to free rotation around the C₇-C₁₁ bond, in an alternate embodiment of the invention this same transformation may also be accomplished by reduction of the C₁₁-C₁₂ double bond on 1 and introduction of the carboxylic acid functionality at C₁₃. It shall be further recognized that these transformations do not have to be carried out in any particular order, that is, it may be desirable to first introduce the oxygen bearing functionality and then carry out the double bond reduction or vice versa, and indeed, that by suitable selection of reagents it may be possible to reduce the double bond and introduce the oxygen in a single step.

In another embodiment of the invention, the exocyclic double bond in amorpha-4,11-diene 1 is functionalized by a mechanism selected from regioselective epoxidation and regioselective dihydroxylation.

IV. a.) Conversion of Amorpha-4,11-diene 1 to DHAA 3 via Compound 5

In one embodiment of the current invention, amorphadiene, (compound 1 in Table 1), is converted to 3 via compound 5 according to Scheme 1. The alcohol moiety of compound 5 is subsequently oxidized to a carboxylic acid moiety, thereby preparing DHAA 3.

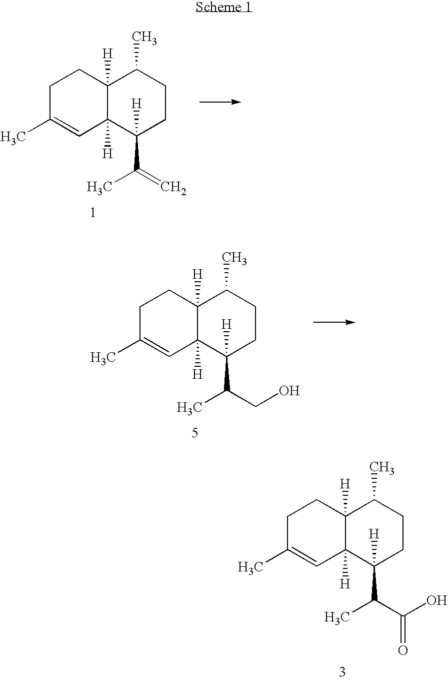

There are a variety of methods to perform the conversion of 1 to 5. In an exemplary embodiment, the reactants can be chosen so as to react selectively with an exocyclic alkene moiety over an endocyclic alkene moiety. The reactants can also be chosen so that the hydroxy group is introduced in an anti-Markovnikov orientation.

In another exemplary embodiment, the conversion of the exocyclic alkene moiety to an alcohol moiety in an anti-Markovnikov orientation is accomplished by a hydroboration reagent. There are a variety of hydroboration reagents of use in the invention. These compounds are described in a variety of publications, including: BORANES IN ORGANIC CHEMISTRY, H. C. Brown, Cornell University Press, 1972; ORGANIC SYNTHESES VIA BORANES, H. C. Brown, John Wiley & Sons Inc, 1975; ORGANOBORANES FOR SYNTHESES (ACS Symposium Series), P. V. Ramachandran and H. C. Brown, American Chemical Society, 2001; and Yadav, J. S. et al., ARKIVOC, 3: 125-139 (2003). In an exemplary embodiment, the hydroboration reagent is borane. In another exemplary embodiment, the hydroboration reagent is borane with a coordinated stabilizing species. Examples of coordinated stabilizing species include, but are not limited to, ethers, sulfides and amines.

In another exemplary embodiment, the hydroboration reagent is monoalkylborane, such as ethylborane. In yet another exemplary embodiment, the hydroboration reagent is dialkylborane, such as diethylborane. In still another exemplary embodiment, the hydroboration reagent is a monocycloalkylborane, such as cyclohexylborane. In some exemplary embodiments, the hydroboration reagent is a dicycloalkylborane, such as dicyclohexylborane or a more complex species such as catecholborane or 9-borabicyclo[3.3.1]nonane (BBN). In another exemplary embodiment, the hydroboration reaction is carried out in the presence of an organometallic catalyst resulting in enhanced regio- and stereoselectivity. Examples of such organometallic species include Rh and Ir compounds (see Evans, D. A. et al., J. Am Chem Soc, 114: 6671-6679 (1992) or Burgess, K. et al., J. Org. Chem., 53: 5179-5181 (1988)).

The conversion of 5 into DHAA 3 may be accomplished in one step using reactants chosen so as to oxidize a primary alcohol to a carboxylic acid. Exemplary oxidizing reagents are described in ADVANCED ORGANIC CHEMISTRY, March, J., John Wiley & Sons, 1992, 4th Ed. These oxidizing agents include chromic acid (ORGANIC CHEMISTRY, Wade, L. G., Prentice Hall, 2003, 5th Ed., Chapters 10, 11, and 20), Jones reagent (a solution of diluted chromic acid in acetone) (Wade, L. G., supra; Yadav, J. S. et al., ARKIVOC, 3: 125-139 (2003)), permanganate (Rankin, K. N. et al, Tetrahedron Lett. 39:1095 (1998)), nitric acid (OXIDATIONS IN ORGANIC CHEMISTRY, Hudlicky, M., American Chemical Society, 1990; COMPREHENSIVE ORGANIC TRANSFORMATIONS, Larock, R. C., VCH, 1989, p. 93), H₅IO₆/CrO₃ (Zhao, M. et al., Tetrahedron Lett. 39: 5323 (1998)), 2,2,6,6-tetramethyl-1-piperidinyloxy (TEMPO)/NaClO/NaClO₂ (Zhao et al., J. Org. Chem., 64:2564 (1999)), pyridinium dichromate (PDC)/dimethylformamide (Corey, E. J. et al., Tetrahedron Lett., 399 (1979)), Na₂WO₄,/aqueous H₂O₂/phase transfer catalyst (Noyori et al., J. Am. Chem. Soc., 119: 12386 (1997)), trichloroisocyanuric acid/RuCl₃ (Ikanuka et al., Org. Proc. Res. Dev., 8: 931 (2004)). In an exemplary embodiment, the oxidizing reagent is Jones reagent.

IV. b.) Conversion of Amorpha-4,11-diene 1 to DHAA 3 via 5 and 7

Alternatively, the oxidation of 5 to 3 can be accomplished in two stages, involving the oxidation of 5 to the corrresponding aldehyde 7 and the subsequent oxidation of 7 to 3.

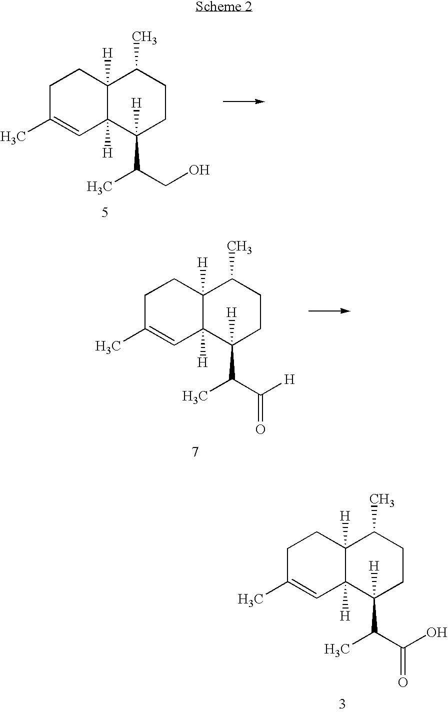

Examples of oxidizing reagents for the transformation of an alcohol to an aldehyde are listed in COMPREHENSIVE ORGANIC TRANSFORMATIONS: A GUIDE TO FUNCTIONAL GROUP PREPARATIONS, 2^{nd} ED, R. C. Larock, Wiley, 1999, pp. 1234-55, while reagents to accomplish the oxidation of the aldehyde to the carboxylic acid are listed in METHODS FOR THE OXIDATION OF ORGANIC COMPOUNDS, A. H. Haines, Academic Press, 1988, pp. 241-43 and 423-428; Larock, loc. cit., pp. 838-840; Hudlicky, loc. cit., pp. 174-180; Dalancale et al, J. Org. Chem., 51: 567 (1986); and Uskokovic et al, J. Org. Chem. 58: 832, (1993).

IV. c.) Conversion of Amorpha-4,11-diene 1 to DHAA 3 via Compound 6

In an alternate embodiment of the invention amorpha-4,10-diene 1 is converted to DHAA 3 through one of the methods outlined in Scheme 3.

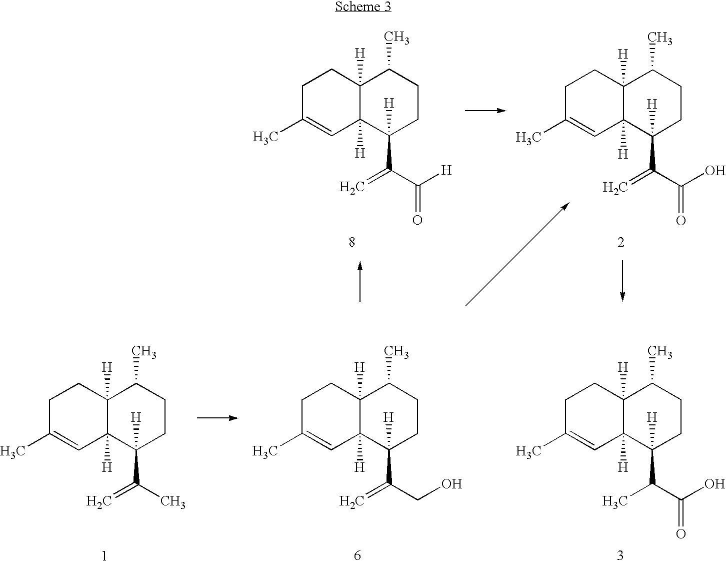

According to Scheme 3 an alcohol functionality is introduced at C₁₃ of 1, without affecting the C₁₁-C₁₂ double bond, affording a compound such as amorpha-4,11-diene-13-ol 6. Compound 6 can then be converted into DHAA 3 via a one-step oxidation to 2, followed by reduction of the C₁₁-C₁₂ double bond to afford 3. In an alternative embodiment, 6 can be converted to 2 via a two-step transformation, in which 6 is first converted to the aldehyde 8, and then further oxidized to afford 2. The reduction of the double bond can be performed at different steps within the overall synthesis. Considerations in choosing a synthetic route include cost, commercial availability of starting materials, ease of reagent handling, environmental friendliness of the overall process, yields, stereoselectivity and ease of purification of intermediates and products. Keeping those considerations in mind, it might be beneficial to synthesize DHAA 3 by a sequence selected from 6→5→3, 6→2→3, 6→5→7→3, 6→8→7→3 and 6→8→2→3.

The conversion of 1 to 6 may be achieved by a variety of means. Broaddus et al (U.S. Pat. No. 3,658,925; J. Am. Chem. Soc., 94: 4298-4306 (1972)) prepared compounds equivalent to 3 via treatment with n-butyllithium-tetramethylethylene-diamine (TMEDA) complex, followed by air oxidation of the resulting C₁₁-C₁₃ anion. U.S. Pat. No. 5,574,195 to Chastain et al. teaches that an efficient means of accomplishing this same transformation is to quench the intermediate allylic anion with a boric acid ester, followed by oxidation of the resulting borate with hydrogen peroxide.

An indirect method of performing said transformation involves an “ene” halogenation to give compound 9 (X=halogen) and then to take advantage of the known instability towards hydrolysis of such compounds to effect the exchange of the halogen for an OH group to afford a compound such as 6. Reagents which will accomplish said “ene” halogenation include, but are not limited to those described in March, loc. cit, pp. 694-697 and others, such as the Vilsmeier reagent/H₂O₂ (Li et al, Tetrahedron Asymmetry, 9: 2607 (1996)), calcium hypochlorite/CO₂ (Wolinski et al, J. Org. Chem., 47: 3148 (1982)) or CeCl₃/NaClO (Massanet et al, Tetrahedron Lett. 44: 6691-6693 (2003)).

In yet another embodiment of the invention, one may bypass the alcohol stage, going directly from compound 9 to compound 8 using any of the numerous reagents for this purpose listed in March, loc. cit. p. 1193.

IV. c.) i.) Synthesis of DHAA 3 from Amorphadiene 1 via Compounds 10, 11 and 6

In one embodiment of the invention, the transformation of compound 1 into compound 6 is achieved via the intermediates 10 and 11 (Scheme 4, Example 3.2.1 and 3.2.2).

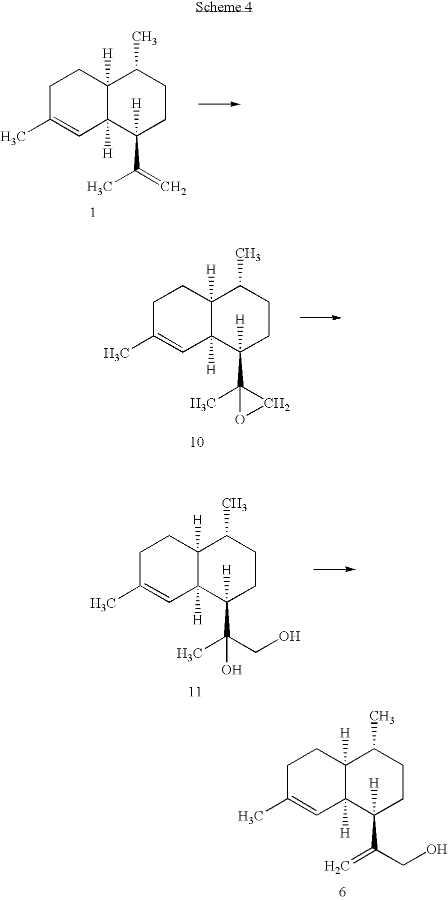

In Scheme 4 the exocyclic (C11-C12) alkene moiety of compound 1 is converted to an epoxide function by reaction with a suitable reagent, thus providing intermediate 10. In a second step the epoxide function in intermediate 10 is opened hydrolytically to give the diol intermediate 11. Finally, the selective removal of water involving the tertiary hydroxyl group located at C11 in intermediate 11 restores the double bond and affords the intermediate 6. The intermediate 6 is then converted to DHAA 3 as described herein. While this sequence involves more synthetic steps than other routes to 6, it is known to those skilled in the art that the reactions involved typically give very high yields. Alternatively, compound 10 could be isomerized directly to compound 6. Compounds 10 and 11 are novel molecules.

Reagents to accomplish the transformation of 1 to 10 include, but are not limited to those listed in March, loc. cit. pp. 826-829 and references cited therein. Examples for epoxidation reagents are given above. In an exemplary embodiment, treatment of amorpha-4,11-diene 1 with a catalytic amount of the metalloporphyrin Mn(2,6-C12TPP)Cl and a stoichiometric excess (5 equivalents) of hydrogen peroxide as the oxygen source results in the preferential formation of the corresponding exocyclic monoepoxide 10 (Example 3.2.1).

Following selective formation of the desired monoepoxide of amorpha-4,11-diene, compound 10 is opened to give the diol 11. The hydrolytic opening of epoxides to afford 1,2-diols is a well established reaction, that can be accomplished under both acidic or basic catalysis in a variety of solvent systems. Examples are given in March, loc. cit., pp. 376-377. In an exemplary embodiment the diol 11 is prepared by treating the epoxide 10 with concentrated sulfuric acid (Example 3.2.2.).

It is known in the art that the ease of dehydration increases with α-branching, to the extent that tertiary alcohols often dehydrate spontaneously in the presence of trace amounts of acid to initiate the reaction. Therefore, one skilled in the art would expect that of the two alcohol functions present in intermediate 11, the tertiary hydroxy group (at C11) should be eliminated with greater ease than the primary hydroxy group (at C12), thus providing a route for the synthesis of intermediate 6.

Compound 11 can be further converted to amorpha-4,11-diene-13-ol 6. Under certain reaction conditions, the tertiary hydroxy group in compound 11 eliminates spontaneously and the diol 11 is found mixed with the final target 6. The dehydration of alcohols to give an alkene can be accomplished by a variety of reagents and under different reaction conditions, such as those mentioned in March, loc. cit., pp. 1011-1012. These methods can be used to drive the elimination reaction to completion in favor of compound 6.

IV. c.) ii.) Preparation of DHAA 3 from Amorphadiene 1 via Compounds 11 and 6

In another embodiment of the invention, the transformation of compound 1 to compound 6 may be achieved via the intermediate 11 (Scheme 5).

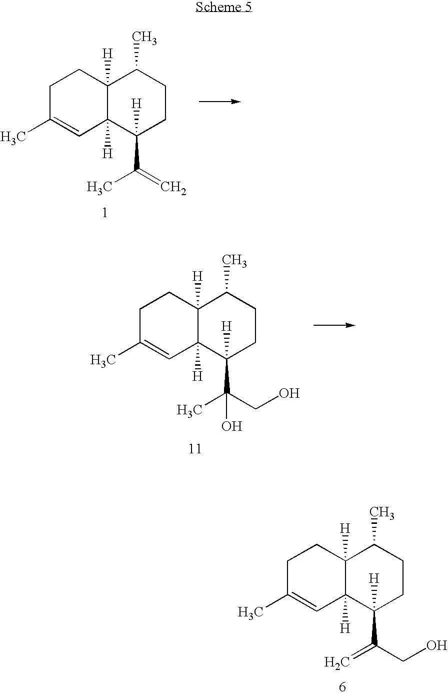

In a first step the exocyclic (C11-C12) alkene moiety of compound 1 is dihydroxylated by reaction with a suitable reagent, thus providing intermediate 11. In a second step, compound 11 is converted to the intermediate 6 by elimination of the tertiary hydroxy group at C11 in intermediate 11, to form compound 6 as described above. Compound 6 is then converted to DHAA 3 as described herein.

It shall be evident to one skilled in the art that the substrate 1 contains two alkene functions amenable to dihydroxylation and that the success of the proposed sequence of reactions leading from 1 to 6 via intermediate 11 depends on achieving the site-selective dihydroxylation of the exocyclic (C11-C12) alkene bond in the presence of the endocyclic (C4-C5) bond. In an exemplary embodiment said conversion is accomplished using an oxidant, which is a member selected from osmium tetraoxide (OsO₄) and ruthenium tetraoxide (RuO₄). In another exemplary embodiment amorphadiene 1 is treated with a catalytic amount of osmium tetroxide and the co-oxidant N-methylmorpholine-N-oxide (NMO) to afford the 1,2-diol 11, as outlined in Scheme 6. 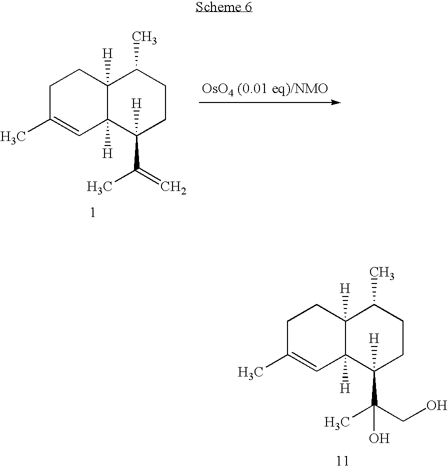
IV. d.) Conversion of Compound 6 to DHAA 3

The conversion of compound 6 to DHAA 3 can be accomplished through different synthetic routes. In an exemplary embodiment the hydroxyl group in compound 6 is first oxidized to afford artemisinic acid 2, and the exocyclic double bond in compound 2 is then reduced to form DHAA 3. In another exemplary embodiment, the exocyclic double bond in compound 6 is first reduced to form compound 5. The hydroxyl group in compound 5 is then oxidized to afford DHAA 3.

IV. d.) i.) Reduction of the Exocyclic Alkene

The reduction of the C₁₁-C₁₂ alkene bond in a compound, such as 2 and 6, can be accomplished by a variety of methods whereby two hydrogen atoms are added across this double bond without affecting the C₄-C₅ double bond and other functional groups which may be present in the substrate such as an alcohol (see compound 6), an aldehyde (see compound 8) or a carboxylic acid moiety (see compound 2) (Scheme 7).

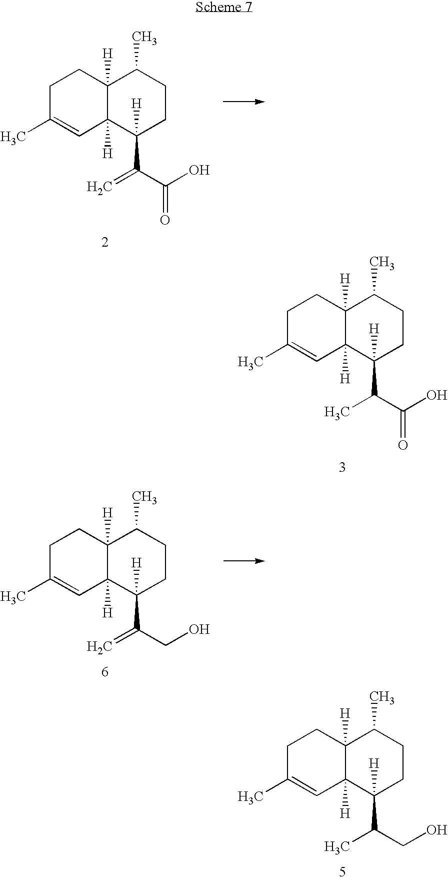

Reagents to accomplish the transformations in Scheme 7 are given, for example, in March, loc cit p. 771. The reduction of 2 to 3 has previously been carried out using “nickel boride”, produced in situ by reaction of a nickel (II) salt with sodium or lithium borohydride (for example, see Xu et al, Tetrahedron, 42:819 (1986)). This process suffers from several drawbacks, most notably: (a) the reduction requires the use of a super-stoichiometric excess of the sodium borohydride (with the obvious problems of handling, workup and cost when working on a larger scale); (b) the reduction cannot be efficiently carried out directly in the presence of a carboxylic acid moiety (i.e. compound 2), as the reducing agent is partially consumed through formation of a mono- or tris-substituted acyloxyborohydride. Thus, the method requires protection of the carboxylic acid moiety for example as an ester such as compound 13, where R is a methyl group, or another alkyl moiety; and (c), although complete stereoselectivity has been claimed for this transformation (for example, in Jung et al., Synlett: 74 (1990), others (see Haines et al., Synlett 491, (1992) and references therein) have reported that those results cannot be reproduced and the product of such a reaction is always an approximately 85:15 mixture of the desired (R) epimer (compound 13, R═H in Table 1) and the undesirable (S) epimer (not shown). Variants of this process have been described (for example, in U.S. Pat. No. 4,992,561, to Roth et al.) but do not address the described drawbacks.

An alternate approach is regio- and enantioselective catalytic hydrogenation, a technique developed by Knowles and Noyori (Knowles et al., J. Am. Chem. Soc., 99: 5946 (1977); Noyori et al. J. Am. Chem. Soc., 102: 7935 (1980)). In this technique, a chiral transition metal catalyst is used to achieve enantioselective hydrogenation of alkenes without covalently altering other functional groups such as those found in compounds 6, 8 or 2. In this instance the selected catalyst must not only distinguish between the alkene moiety and other functional groups, but also between the endocyclic and the exocyclic alkene moieties. In an exemplary embodiment, a BINAP-Ru calalyst is used to convert compound 6 to compound 5. In another exemplary embodiment the catalytic hydrogenation is performed in the presence of Wilkinson's catalyst. Using these approaches the desired (R)-enantiomer is formed without producing significant amounts of the undesired (S)-enantiomer.

In an exemplary embodiment, compound 2 is provided through a biological source.

V. Large-Scale Preparation of DHAA 3

In another exemplary embodiment, the method of the current invention provides DHAA 3 in an amount of at least one kilogram. Large scale production of DHAA can be achieved by utilizing methods currently known in the art. For example, large scale hydroboration can be accomplished through suitable modification of the method described in Ripin et al., Org. Proc. Res. Dev., 7: 115-120 (2003). Adjustments to the reported procedure include the substitution of the reported alkene substrate with amorpha-4,11-diene 1.

In addition, large scale oxidation of primary alcohols to carboxylic acids can be accomplished. In general, the conditions for a small-scale oxidation of primary alcohols to carboxylic acids are proportional to the conditions for a large-scale oxidation of primary alcohols to carboxylic acids. Minor adjustments, such as determining the proportions necessary to convert small scale reaction conditions to large scale reaction conditions, are required. These adjustments are well within the knowledge of one of skill in the art.

Large scale enantioselective catalytic hydrogenations are also well known in industry, and numerous examples are described, for example, in ASYMMETRIC CATALYSIS ON INDUSTRIAL SCALE, H. U. Blaser and E. Schmidt, Wiley-VCH, 2004.

VI. Synthesis of Artemisinin 4 from DHAA 3

In another aspect, the invention provides a method of preparing artemisinin 4: 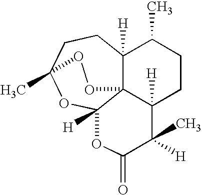
said method comprising, (a) converting DHAA 3 or an esterified derivative thereof to an oxidized species using an oxidation procedure, wherein the oxidation procedure is a member selected from photochemical oxidation and non-photochemical oxidation, (b) subjecting the product of step (a) to an acid or metal catalyzed rearrangement reaction, (c) oxidizing the product of step (b), and (d) subjecting the product of step (c) to two acid catalyzed cyclizations in order to produce artemisinin 4.

In an exemplary embodiment the DHAA 3 used to prepare artemisin 4 is prepared from amorpha-4,11-diene 1 by one of the methods described herein.

In another exemplary embodiment, the DHAA 3 used to prepare artemisin 4 is derived from a biological source. In another exemplary embodiment DHAA 3 is prepared from artemisinic acid 2. In a further exemplary embodiment 2 is isolated from a biological source. In another exemplary embodiment the organism which produces either DHAA 3 or artemisinic acid 2 is obtained through recombinant technology.

VI. a.) Oxidizing DHAA 3 Using Photochemical Oxidation

In an exemplary embodiment, the conversion of dihydroartemisinic acid 3 to artemisinin 4 comprises subjecting dihydroartemisinic acid 3 to a photochemical oxidation.

In an exemplary embodiment, said photochemical oxidation comprises contacting, with light, a mixture comprising dihydroartemisinic acid 3, oxygen and a singlet oxygen photosensitizer. In an exemplary embodiment the photosensitizer is a member selected from methylene blue and rose Bengal.

In this reaction DHAA 3 or its ester 13 (R═H) is converted to dihydroartemisinic acid hydroperoxide, which has a structure according to the following formula:

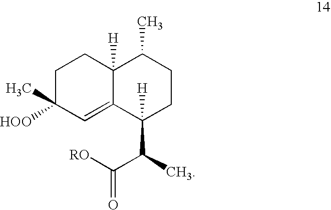

In compound 14, R is a member selected from H, substituted or unsubstituted alkyl and substituted or unsubstituted heteroalkyl.

Compound 14 is formed through the addition of singlet oxygen via photooxidation in an organic solvent in the presence of a photooxidizer and light. Examples of this photooxidative reaction are described in U.S. Pat. No. 4,992,561 to Roth et al., as well as in Acton et al., J. Org. Chem. 57: 3610-3614 (1992), both of these references are herein incorporated by reference. Compound 14 can be further subjected to an acid catalyzed oxidation-ring closure reaction in order to prepare artemisin 4.

In an exemplary embodiment, the carboxylic acid moiety on dihydroartemisinic acid 3 is converted to a carboxylic acid derivative moiety prior to the photochemical oxidation. The carboxylic acid derivative moiety is a member selected from esters, acid chlorides, acid bromides, acid anhydrides, amides, thioacids, and thioesters. In an exemplary embodiment the carboxylic acid derivative moiety is an ester.

VI. b.) Preparation of Artemisinin 4 using Non-photochemical Oxidation

The aforementioned photochemical oxidation step represents a bottleneck in the manufacture of artemisinin 4 in an amount of at least one kilogram. Large-scale photochemical reactions are usually performed with one or more macro-scale lamps immersed in the reaction vessel. In most cases, it takes considerable effort to scale-up a successful lab-scale reaction to its industrial counterpart. Issues involved include the scalability of light sources, heat and mass transfer in the processes, reduced efficiency at greater distances from the lamp and safety concerns (e.g. explosions caused by excess heat). Many photochemical reactions proceed via a free-radical mechanism. If the radicals, which are formed near the light sources, do not diffuse quickly to react further with other species, they are likely to recombine, generating heat instead of desired products. Radical recombination also reduces the quantum efficiency of the overall process.

Non-photochemical generation of excited state oxygen species has been the subject of several investigations including the in depth study of Aubry, J. Am Chem. Soc. 107: 5844-5849 (1985), describing the decomposition of hydrogen peroxide to form “singlet oxygen” as determined indirectly by chemical trapping with electron rich dienes. Recent refinements in the non-photochemical methods of converting hydrogen peroxide to singlet oxygen are described in the literature and employ calcium Aubry, et al, Chem. Commun. 599-600 (1998), Aubry, et al, J. Org. Chem. 67: 2418-2423 (2002), molybdenum Tetrahedron Lett. 43: 8731-8734 (2002), J. Am. Chem. Soc. 126: 10692-10700 (2004) and lanthanide Aubry, et al, Chem. Eur. J. 9: 435-441 (2003), Aubry, et al, Chem. Commun., 927-929 (2005) metal salts to catalyze this reaction. In addition to forming cycloadducts with electron rich dienes, these reagent systems are known to produce diketones and allylic hydroperoxides.

In an exemplary embodiment, the conversion of dihydroartemisinic acid 3 or its ester 13 to artemisinin 4 comprises, converting dihydroartemisinic acid 3 or an esterified derivative thereof to an oxidized species using a non-photochemical oxidation procedure.

In an exemplary embodiment the oxidized species is the hydroperoxide 14, wherein R is a member selected from H, substituted or unsubstituted alkyl and substituted or unsubstituted heteroalkyl.

In an exemplary embodiment, the hydroperoxide is generated in the presence of a member selected from a peroxide, an endoperoxide and an ozonide.

In an exemplary embodiment, said non-photochemical oxidation is accomplished in the presence of hydrogen peroxide and a metal catalyst, which converts hydrogen peroxide to singlet oxygen. In an exemplary embodiment, the metal in the metal catalyst is a member selected from lanthanum, cerium, molybdenum, calcium, tungsten, scandium, titanium, zirconium and vanadium. Those metals may be used in the form of a salt or an oxide. In a preferred embodiment the metal catalyst is sodium molybdate. Other examples for catalysts include lanthanum nitrate, calcium hydroxide, and sodium tungstate.

In yet another exemplary embodiment, the metal catalyst is supported on a solid inorganic or organic medium which is a member selected from alumina, silica, a zeolite and an organic polymer.

IV. c.) Preparation of Artemisin 4 from the Hydroperoxide 14

In an exemplary embodiment the product of the photochemical or non-photochemical reaction, for example compound 14, is subjected to an oxidation-ring closure reaction comprising (i) subjecting the product of the photochemical oxidation or the non-photochemical oxidation to an acid or metal catalyzed rearrangement, (ii) oxidizing the product of the rearrangement reaction, and (iii) subjecting the oxidized product to two acid catalyzed cyclizations in order to produce artemisinin 4.

Examples of acid catalyzed oxidation-ring closure reactions include oxidation via triplet oxygen in air (U.S. Pat. No. 4,992,561 to Roth et al.) or via a metal catalyst (U.S. Pat. No. 5,310,946 to Haynes), or by various other methods, leading to the formation of an enol ketone (compound 18, X═H in Table 1). Compound 18 then rapidly autooxidizes to a keto-aldehyde hydroperoxide intermediate (compound 16 in Table 1). This intermediate is immediately closed in an acid catalyzed process, beginning with the endocyclic hydroperoxide bridge formation and finishing with the nucleophilic attack of the carbonyl carbon and the substitution of the hydroxyl in the carboxylic acid, to form artemisinin 4.

In an exemplary embodiment, the metal catalyst in step (i) (metal catalyzed rearrangement) is a copper salt. In another exemplary embodiment, the copper salt is a member selected from copper (II) trifluoromethanesulfonate, copper (II) sulfate, copper (II) acetate, copper (II) acetylacetonate, and copper (II) chloride.

In another exemplary embodiment, the acid in step (iii) (acid catalyzed cyclization) has a pKa of between 5 and −20. In another exemplary embodiment, at least one of said acids is a protic acid. In a further exemplary embodiment the protic acid is a member selected from acetic acid, trifluoroacetic acid, methanesulfonic acid, citric acid, p-toluenesulfonic acid and oxalic acid.

In another exemplary embodiment the acid in step (iii) is a substance comprising a polymeric backbone or matrix containing acidic functional groups. In an exemplary embodiment the polymeric backbone or matrix is a member selected from styrene-divinylbenzene compolymer, an acrylate, a methacrylate, a phenol-formaldehyde condensate, an epichlorohydrin amine condensate and a perfluorinated ionomer. In another exemplary embodiment, the acidic functional groups on the polymeric backbone or matrix are members selected from sulfonates, phosponates and carboxylic acids. In yet another exemplary embodiment, the acid in step (i) is an acidic resin. In a further exemplary embodiment, the acidic resin is sulfonated polystyrene, such as DOWEX 50WX8-200.

IV. d.) Preparation of Artemisinin 4 using Carboxylic Acid Derivatives

In another exemplary embodiment, the conversion of dihydroartemisinic 3 acid to artemisinin 4 comprises, (i) converting the carboxylic acid moiety on dihydroartemisinic acid 3 to a carboxylic acid derivative moiety, wherein said carboxylic acid derivative moiety is a member selected from esters, acid chlorides, acid bromides, acid anhydrides, amides, thioacids, and thioesters; (ii) subjecting the product of step (i) to an oxidation, wherein the oxidation is a member selected from a photochemical oxidation and a non-photochemical oxidation, (iii) subjecting the product of step (ii) to an acid or metal catalyzed rearrangement reaction; (iv) oxidizing the product of step (iii); and (v) subjecting the product of step (iv) to two acid catalyzed cyclizations in order to produce artemisinin 4.

Converting DHAA 3 to a carboxylic acid derivative prior to oxidation can significantly enhance yields or improve purity of the final product. In a preferred embodiment DHAA 3 is converted to its corresponding ester 13 prior to subjecting it to either a photochemical oxidation or a non-photochemical oxidation. During the synthesis of artemisinin 4, the ester undergoes the same sequence of reactions as DHAA 3 except that in the final ring closure the leaving group is an alkoxy group instead of a hydroxyl group. It shall be apparent to one skilled in the art that many methods for generating appropriate esters are available.

Alternatively the ester of DHAA 3 can be prepared by converting the carboxylic acid moiety on artemisinic acid 2 to an ester functionality and reducing the exocyclic double bond in the resulting artemisinic acid ester 12 to afford DHAA ester 13.

Other intermediates in the synthetic routes for the preparation of artemisinin 4 maybe converted to a corresponding ester. For instance, the hydroperoxide 14 (R═H) can be methylated with diazomethane to form the hydroperoxide methyl ester (compound 14, R═CH₃) (U.S. Pat. No. 5,310,946). This reaction results in a modification of the solubility of the compound in organic solvents and a leaving group slightly preferred to the hydroxyl group. Since this modification affects the leaving group only, the subsequent oxygenation via triplet oxygen leads once again to the formation of artemisinin 4. It shall be apparent to one skilled in the art that alternate methods for generating said ester can be used, as long as the reaction conditions are mild enough to not affect the highly reactive hydroperoxide moiety.

Alternatively, it is likely that the formation of an acid chloride from the corresponding carboxylic acid would lead to a leaving group preferable for the ring closure chemistry. For example, compound 13 (R═H) could be reacted with thionyl chloride at a temperature of 0-50° C. to form the corresponding acid chloride. Alternatively, 13 could be reacted with PCl₃ at 0-150° C. to form the acid chloride. Alternatively, 13 could be reacted with PCl₅ at 0-50° C. to form the acid chloride.

Compound 13 (R═H) or its corresponding acid chloride could alternatively be converted to compounds with other functional groups such as esters, amides, acid anhydrides, thioesters and thioacids that provide suitable leaving groups and that may facilitate the ring closure chemistry, leading to higher yields. Alternatively, the hydroperoxide analogs, such as compound 19 could be converted to suitable carboxylic acid derivatives (compound 20 in Table 1) to produce a starting material for the synthesis of artemisinin. In 20, Y represents a carboxylic acid moiety derivative and is a member selected from amides, acid anhydrides, thioesters and thioacids. Methods of converting carboxylic acid moieties to carboxylic acid moiety derivatives, as well as methods of converting acid chloride moieties to carboxylic acid moiety derivatives are known in the art (ORGANIC CHEMISTRY, L. G. Wade, Prentice Hall, 2003, 5th Ed., Chapters 20 and 21).

In another exemplary embodiment, artemisinin 4 is synthesized by the synthetic route outlined in Scheme 8.

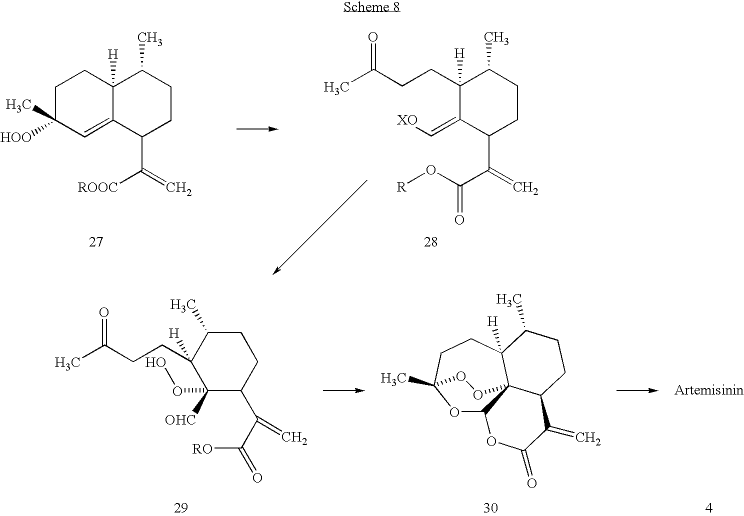

The synthesis starts with artemisinic acid 2, which is converted to the hydroperoxide 27 (R═H) by either photochemical oxidation or non-photochemical means using hydrogen peroxide and an appropriate metal catalyst. The hydroperoxide 27 undergoes protic acid or Lewis acid (e.g., metal salt) catalyzed rearrangement to enol 28, X═H, which is rapidly oxidized by molecular oxygen to give keto-aldehyde hydroperoxide intermediate 29. This intermediate is immediately closed in an acid catalyzed process, beginning with the endocyclic hydroperoxide bridge formation and finishing with the nucleophilic attack of the carbonyl carbon and the substitution of the hydroxyl in the carboxylic acid, to form deshydroartemisinin, 30 which is converted to artemisinin 4 by diastereoselective hydrogenation.

An analogous process occurs for esters of artemisinic acid 12 (R=alkyl) which undergo the same sequence of reactions except that in the final ring closure the leaving group is an alkoxy group instead of a hydroxyl group.

In an exemplary embodiment artemisinin 4 is synthesized by converting amorphadiene 1 to a compound comprising an alcohol moiety and having the formula and stereochemistry shown:

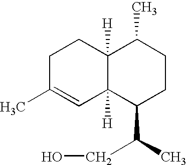

The method further comprises oxidizing the alcohol moiety to a carboxylic acid moiety, thus producing dihydroartemisinic acid 3 with the desired (R)-stereochemistry at C11 (compound 13, R═H).

In an equivalent sequence amorpha-4,11-diene 1 is converted to compound 6, which is in turn enantioselectively reduced to a compound having the structure shown above, which is further treated as indicated to form dihydroartemisinic acid 3.

In an additional embodiment the compound of formula 6 is first oxidized to form artemisinic acid 2 and the latter is enantioselectively hydrogenated to afford dihydroartemisinic acid with the desired (R)-stereochemistry at C11 (compound 13, R═H).

Finally, the method further comprises converting said dihydroartemisinic acid 3 or it's ester, each prepared by one of the methods described above, to artemisinin 4, thereby preparing said artemisinin.

Alternatively, artemisinic acid 2 or its ester 12 is converted to artemisinin 4 by subjecting them to either a photochemical oxidation or a non-photochemical oxidation and subjecting the product to an oxidation-ring closure reaction to give deshydroartemisinin, which is converted to artemisinin 4 by diastereoselective hydrogenation.

In an exemplary embodiment the artemisinic acid 2, used in any of the above conversions is derived from a biological source.

VII. Synthesis of Artemisinin Analogs from Amorpha-4,11-diene 1

In another aspect, the invention provides a method of preparing an artemisinin analog. This method comprises (a) converting amorpha-4,11-diene 1 to a compound comprising an alcohol moiety and having the formula: 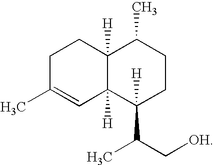
The alcohol moiety on the product of step (a) is then (b) oxidized to an aldehyde moiety, thus producing a dihydroartemisinic aldehyde (compound 7 in Table 1) having a structure according to

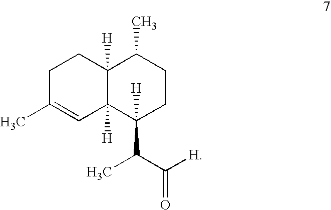

Methods of oxidizing primary alcohols to aldehydes involve oxidizing agents such as pyridinium chlorochromate (PCC), a complex of chromium trioxide with pyridine and HCl (ORGANIC CHEMISTRY, L. G. Wade, Prentice Hall, 2003, 5th Ed., Chapter 11). The aldehyde moiety on the product of step (b) is then (c) converted to an alcohol moiety through the addition of a nucleophile, thereby producing a compound having a structure according to 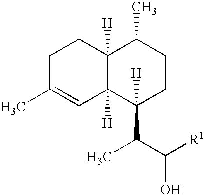
in which R¹ is a member selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

This reduction can be accomplished by a variety of reducing agents. In an exemplary embodiment, the reducing agent is a Grignard reagent which comprises an R¹ moiety (ORGANIC CHEMISTRY, L. G. Wade, Prentice Hall, 2003, 5th Ed., Chapter 10). The product of step (c) can be a leaving group suitable for ring closure. Therefore, the product of step (c) is then (d) subjected to a photochemical oxidation or a non-photochemical oxidation. The product of step (d) is then (e) subjected to an oxidation-ring closure reaction, thus producing said artemisinin analog (compound 22 in Table 1), wherein said artemisinin analog has a structure according to

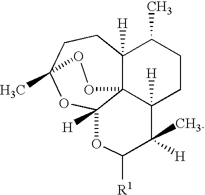

Several of the conversions above are discussed more fully in Haynes, et al., Synlett: 481 (1992)). The content of this document is herein incorporated by reference.

In an alternate embodiment amorphadiene 1 is converted into 6, which is then oxidized to the corresponding aldehyde 8, which is enantioselectively hydrogenated to give 7, which is treated as described in the preceding paragraph to produce an artemisinin analog.

### EXAMPLES

The following examples are provided by way of illustration only and not by way of limitation. Those of skill in the art will readily recognize a variety of non-critical parameters that could be changed or modified to yield essentially similar results.

In the examples below, unless otherwise stated, temperatures are given in degrees Celsius (° C.); operations were carried out at room or ambient temperature, “rt,” or “RT,” (typically a range of from about 18-25° C.); evaporation of solvent was carried out using a rotary evaporator under reduced pressure (typically, 4.5-30 mm Hg) with a bath temperature of up to 60° C.; the course of reactions was typically followed by thin layer chromatography (TLC) and reaction times are provided for illustration only; melting points are uncorrected; products exhibited satisfactory ¹H-NMR and/or microanalytical data; yields are provided for illustration only; and the following conventional abbreviations are also used: mp (melting point), L (liter(s)), mL (milliliters), mmol (millimoles), g (grams), mg (milligrams), min (minutes), h (hours), RBF (round bottom flask).

### Example 1

1.1. Conversion of 1 to 5

A 250 mL flask equipped with a septum inlet and magnetic stir bar was charged with 50 mmol of BH₃SMe₂ and 18 mL of freshly distilled THF. It was cooled to 0° C. and 115 mmol of cyclohexene was added dropwise. After the mixture was stirred at 0° C. for 1 hour, (C₆H₁₁)₂BH separates as a white solid.

To the (C₆H₁₁)₂BH (solid, 50 mmol) were added 75 mmol of amorphadiene 1. The reaction mixture was stirred at −25° C. for one hour and was then placed in a refrigerator for one day. The trialkyl borane was treated with 50 mL of 3N sodium hydroxide, 7.5 mL of 30% hydrogen peroxide and the reaction mixture was stirred at 25° C. for 5 hours. The product was then extracted with ether and dried over sodium sulfate. The ether was subsequently evaporated. The residue was filtered through silica gel (petroleum ether:ethyl acetate 9:1 used as eluent) to remove the olefin and the cyclohexyl alcohol and then eluted with petroleum ether:ethyl acetate (1:1) to give the pure alcohol, 5.

### Example 2

2.1. Conversion of 5 to 3

Jones reagent was prepared by dropwise addition of sulfuric acid (17 mL) to a cooled solution of CrO₃ (200 mmol) in water (30 mL) and the resulting solution was diluted with water until the total volume of the solution was 60 mL.

The alcohol 5 (65 mmol) was dissolved in acetone (150 mL) and cooled to 0° C. Jones reagent was added dropwise through a dropping funnel over a period of 2 hours until the orange brown color of the reagent persisted. The reaction mixture was stirred for another 2 hours. Ether was then added to precipitate out the chromeous salts. The reaction mixture was filtered and the residue was washed with ether. The organic layer was dried over anhydrous sodium sulfate, concentrated and purified by the addition of 5% aq. sodium hydroxide. The product was washed with ether to remove impurities. The aqueous layer was acidified and extracted with ethyl acetate. The extract was dried over anhydrous sodium sulfate and concentrated to produce pure DHAA, 3.

### Example 3

3.1. Conversion of 1 to 3 via 9, 6 and 5

A 250 mL three-necked flask equipped with a thermometer, condenser and a magnetic stir bar was charged with 50 mmol of calcium hypochlorite and 50 mL of water and stirred vigorously while amorphadiene 1, dissolved in 200 mL of methylene chloride, was added over 30 minutes. Stirring was continued for 3 h while 50 g of dry ice was added in small portions at regular intervals. The thick white slurry was filtered to remove inorganic salts. These inorganic salts were washed with two 25 mL portions of methylene chloride. The filtrate and washes were combined, the aqueous layer was decanted and the organic layer was dried over anhydrous sodium sulfate. The organic layer was filtered to remove the drying agent and concentrated under vacuum to give (9, X═Cl). The chlorine on 9 was hydrolyzed by boiling the concentrate with a 50:50 mixture of dioxane and water to give the unsaturated alcohol 6 after concentration. The unsaturated alcohol 6 was dissolved in 50 mL of methanol. 0.05 mmol of BINAP-Ru catalyst were added and the resulting suspension was stirred at room temperature under 50 psi of hydrogen gas until chromatography indicated complete reaction. The reaction mixture was then filtered to remove the catalyst and concentrated under vacuum to afford the crude alcohol 5. 5 may be further treated as described in Example 2.1 to afford DHAA, 3.

3.2. Conversion of 1 to 3 via 10, 6 and 2

3.2.1. Conversion of 1 to 10

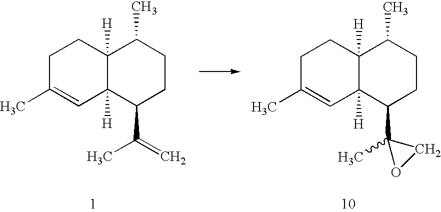

To a solution of amorpha-4,11-diene 1 (55.2 mg; 0.27 mmol) in 2 mL of acetonitrile were added 2.5 mg (1 mol %) of [Mn(2,6-C₁₂TPP)Cl] and 32.6 mg (0.42 mmol) of NH₄AcO. To the reaction was added dropwise a solution of NH₄CO₃H (61.5 mg; 0.82 mmol) and 30% H₂O₂ (ca. 5 equivalents). Vigorous bubbling was observed. The reaction was stirred at room temperature and monitored by TLC (4:1 hexane/ethyl acetate). After 1 hour saturated Na₂S₂O₃ and ethyl acetate were added. The aqueous layer was extracted twice with ethyl acetate. The combined organic extracts were dried over anhydrous K₂CO₃ and concentrated under reduced pressure to yield a brown oil. The crude product was purified by column chromatography to give the amorphadiene-monoepoxide 10, in a 2:1 mixture of two diastereomers. In the ¹H-NMR spectrum of 10 the signals for the (C11-C12) exocyclic double bond (at approximately 4.6 to 4.9 ppm) are absent and the signal for the allylic C6-H is conserved. ¹H-NMR (CDCl₃) (minor diastereomer in brackets) δ: 5.17 [5.50] (br s, 1H), 2.60 [2.40] (d, J=4.5, 1H), 2.83 [2.75] (d, J=4.5, 2H), 2.60 [2.50] (s br, 1H).
3.2.2. Conversion of 10 to 11 and 6

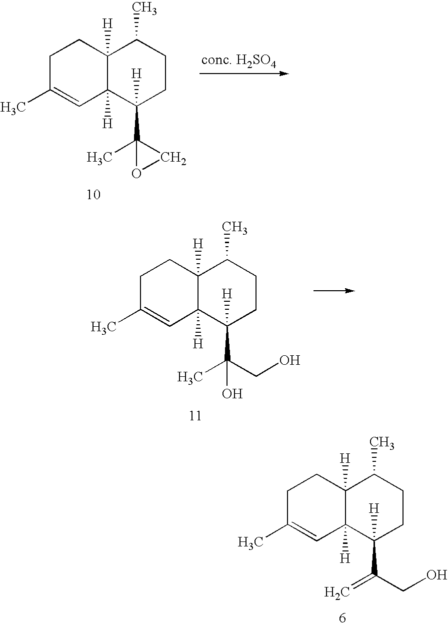

To a solution of 30 mg of 10 in THF/H₂O (4:1) were added 4 drops of concentrated sulfuric acid. The reaction was stirred at room temperature until all of the starting material was consumed. The desired compounds were extracted into ethyl acetate. The organic layer was washed with water and dried over MgSO₄. The solvent was removed by rotary evaporation to yield a light yellow oil, that contained compounds 6 and 11 as the major components, which were separated by column chromatography. TLC (4:1 hexane/ethyl acetate) Rf (allylic alcohol)=0.34 and Rf (diol)=0.14.
3.2.3. Conversion of 6 to 2

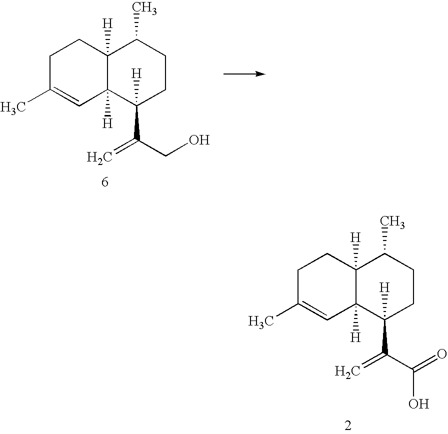

To a solution of 0.2 mmol of 6 in 1 mL acetone at 0° C. were added several drops of Jones Reagent (1.4M CrO₃: 2.2 M H₂SO₄: water). The reaction mixture was allowed to warm to room temperature and stirred until the starting material 6 was consumed. To the reaction was added water and CH₂Cl₂. The organic layer was washed with water and dried over MgSO₄. The solvent was removed in vacuo to yield 2 in quantitative yield (LC/MS). Compound 2 can readily be converted to DHAA, 3, as described by Roth and Acton, J. Chem. Ed., 68:7, 612-613 or by catalytic hydrogenation (Example 4).

### Example 4

4.1. Conversion of 2 to 3

Fifteen mL of methanol were added to a Parr shaker bottle followed by Tris(triphenylphosphine) rhodium (I) chloride (4.2 mg, Wilkinson's catalyst) followed by 71.2 mg of artemisinic acid 2. The suspension was shaken on the Parr apparatus for one hour at 37 psi. NMR shows little or no reaction at this point. The suspension was put back on the Parr apparatus for 12 days at 30-35 psi without shaking. A dried aliquot was analyzed by NMR which showed that the desired isomer of 3 was formed preferentially over the undesired isomer in a ratio of 5.8 to 1 at 62% conversion.

### Example 5

5.1. Conversion of 3 to 4

Dihydroartemisinic acid 3 (40.2 mg, 0.17 mmol) was dissolved in 1 mL of denatured ethanol. To the solution were added 0.1 mL (0.19 mmol) aqueous sodium hydroxide. A white suspension formed. Sodium molybdate dihydrate (8.1 mg) was added followed by portionwise addition of 50% hydrogen peroxide (6 thirty microliter portions were added about twenty minutes apart). Ten minutes after the last addition of hydrogen peroxide the mixture was concentrated by rotary evaporation. The residue was dissolved in ten mL ethyl acetate and five mL water and then acidified to pH 4 with 5% HCl. The phases were separated and the aqueous phase was extracted with 5 mL ethyl acetate. The combined ethyl acetate phases were dried over magnesium sulfate, filtered and concentrated to give 54.3 mg of a colorless oil. Three mL of acetonitrile were added to the oil to produce a suspension. Oxygen gas was bubbled through the suspension and copper (II) trifluoromethanesulfonate (8.0 mg) was added. Thirty minutes after the copper salt was added three mL of methylene chloride was added to the suspension. Eighty minutes later the reaction was concentrated in vacuo. NMR analysis of the crude reaction mixture showed that most of the material is unreacted 3 along with a small amount of artemisinin. The crude artemisinin was dissolved in 10 mL ethyl acetate and washed twice with 10 mL potassium carbonate solution. The ethyl acetate phase was concentrated in vacuo to give 11.7 mg of a colorless oil, which contained approximately 40% artemisinin 4 (NMR, 8% yield). Acidification of the potassium carbonate extracts to pH 4 followed by extraction with ethyl acetate and rotary evaporation gives a recovery of 25.2 mg unreacted dihydroarteminisinic acid 3. The calculated yield based on recovered 3 is about 26%.

### Example 6

6.1. Conversion of 3 to 13 (R═CH₃)

Dihydroartemisinic acid 3 (0.17 mmol) was dissolved in 1.4 mL dimethylformamide and potassium carbonate (0.25 mmol) was added followed by iodomethane. The light yellow suspension was stirred for 20 hours at room temperature and was then diluted with 10 mL water and 10 mL ether. The phases were separated and the aqueous phase was acidified to pH 4 with 5% HCl. The aqueous phase was extracted with 10 mL ether and another 5 mL ether. The combined ether extracts were dried over potassium carbonate, filtered and concentrated to give 95.4 mg of a light yellow liquid. The ester was purified by silica gel column chromatography using 5% ethyl acetate in hexanes as eluent to yield 13, R═CH₃ (36.0 mg 84%) as a light yellow oil.

### Example 7

7.1. Conversion of 13 (R═CH₃) to 4

A solution of deuterated ethanol(ethanol-d⁶) (92.4 mg), deuterium oxide (85.6 mg) and sodium dodecylsulfate (53.3 mg) in 0.50 mL methylene chloride was added to crude 13 (93.6 mg, 78% pure, 0.292 mmol) followed by sodium molybdate dihydrate (10.6 mg). Hydrogen peroxide (50%) was added in three portions (30, 30 and 35 microliters) at t=0, 45 min and 80 min, respectively. After an additional 80 minutes the solution was added to 8 mL of 50% v/v ethyl acetate in hexanes. The solution was concentrated to about 4 milliliters by rotary evaporation and then filtered through a 70-100 micron glass frit and concentrated to give 119 mg oil and white film. Two milliliters of methylene chloride was added and the mixture was cooled in an ice bath. Oxygen gas was bubbled through the solution and copper (II) triflate (7.8 mg) was added and oxygen bubbling was continued. Forty minutes later, a suspension of DOWEX 50WX8-200 resin (sulfonated polystyrene, 50.2 mg) was added and oxygen bubbling was continued for an additional 30 minutes. The suspension was stirred at room temperature for an additional 18 hours and then filtered and concentrated to give 114.3 mg of a brown oil which partially solidified in the freezer. Purification on silica gel using 10% ethyl acetate/hexanes and then 20% ethyl acetate/hexanes as eluents gave 36.1 mg artemisinin as a white solid (34% or 43% yield if corrected for impurities in the starting ester).

### Example 8

8.1. Conversion of Valencene 26 to Valencene-11,12-epoxide

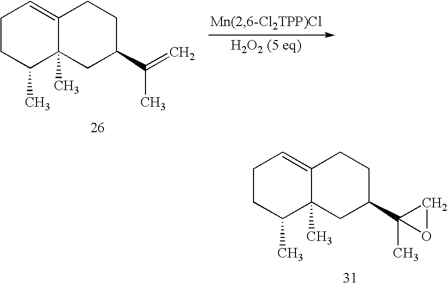

Valencene 26 (53 mg; 0.26 mmol), was dissolved in 1 mL of acetonitrile. To the solution was added 2.2 mg (1 mol %) of [Mn(2,6-C₁₂TPP)Cl] and 32.4 mg (0.42 mmol) of NH₄OAc. To the reaction was added dropwise in portions a solution of NH₄HCO₃ (70 mg; 0.89 mmol) and 30% H₂O₂ (ca. 5 equivalents). Vigorous bubbling was observed. The reaction was stirred at room temperature and monitored by TLC (4:1 hexane/ethyl acetate). After 1 hour saturated Na₂S₂O₃ and ethyl acetate was added. The aqueous layer was extracted 2× with ethyl acetate. The combined organic extracts were dried over anhydrous K₂CO₃ concentrated under reduced pressure to yield a brown oil. The crude product was purified by column chromatography to yield valencene-11,12-epoxide.

### Example 9

9.1. Conversion of Valencene 26 to Valencene-11,12-diol 32

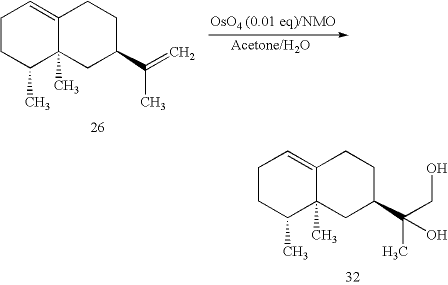

Valencene 26 (225.5 mg; 1.1 mmol) was dissolved in 5 mL of acetone and 1 mL of water. To the solution was added 155 mg (1.32 mmol) of NMO. The solution was cooled to 0° C. Approximately 0.01 equivalents of OsO₄ (4% solution) was added to the reaction. The reaction was allowed to warm to room temperature and stirred at room temperature until the reaction was complete. The reaction was monitored by GC/MS. Upon completion solid sodium bisulfite was added to the reaction mixture. The slurry was stirred at room temperature for approximately one hour. To the quenched solution was added CH₂Cl₂ and water. The layers were separated and the aqueous layer was extracted with additional CH₂Cl₂. The combined organic extracts were dried, filtered through Celite, and concentrated by rotary evaporation to yield crude valencene-11,12-diol.

While this invention has been disclosed with reference to specific embodiments, it is apparent that other embodiments and variations of this invention may be devised by others skilled in the art without departing from the true spirit and scope of the invention.

All patents, patent applications, and other publications cited in this application are incorporated by reference in the entirety.

What is claimed is:

1. A method of regioselectively epoxidizing an exocyclic alkene over an endocyclic alkene; said method comprising:
(a) contacting a substrate, an epoxidation oxidant and a member selected from a metalloporphyrin and a metallosalen.

2. The method according to claim 1, wherein the metal in the metalloporphyrin or the metallosalen is a transition metal.

3. The method according to claim 2, wherein said transition metal is a member selected from chromium, manganese, iron, cobalt, nickel, copper, zinc, ruthenium, and palladium.

4. The method according to claim 1, wherein the porphyrin portion in the metalloporphyrin is a member selected from TPP, TTMPP and TTP.

5. The method according to claim 1, wherein the epoxidation oxidant is a member selected from oxygen, a peroxide, a peracid, a hypochlorite, a peroxydisulfate (S₂O₈²⁻), a dioxyrane, iodosylbenzene (PhIO), and combinations thereof.

6. The method according to claim 5, wherein the peroxide is hydrogen peroxide.

7. The method according to claim 1, wherein the substrate is a member selected from a monoterpene, a sesquiterpene, a diterpene, and a triterpene.

8. The method according to claim 7, wherein the sesquiterpene is a member selected from an amorphane, a valencane, a cadinane, an eremophilane, a guaiane, a germacrane and a eudesmane.

9. The method according to claim 7, wherein the sesquiterpene is amorpha-4,11-diene.

10. A method of regioselectively dihydroxylating an exocyclic alkene over an endocyclic alkene; said method comprising:
(a) contacting a substrate with a dihydroxylation reagent which comprises a transition metal based oxidant (or catalyst).

11. The method according to claim 10, wherein the oxidant is a member selected from osmium tetraoxide (OsO₄) and ruthenium tetraoxide (RuO₄).

12. The method according to claim 10, wherein the dihydroxylation reagent further comprises a co-oxidant for the regeneration of the primary oxidant.

13. The method according to claim 12, wherein the co-oxidant is a member selected from a peroxide, a peracid, a tertiary amine N-oxide, K₃Fe(CN)₆, a chlorite, I₂, a selenoxide and a peroxysulfate (S₂O₈²⁻).

14. The method according to claim 13, wherein the tertiary amine N-oxide is N-methylmorpholine-N-oxide (NMO).

15. The method according to claim 10, wherein the substrate is a member selected from a monoterpene, a sesquiterpene, a diterpene and a triterpene.

16. The method according to claim 15, wherein the sesquiterpene is a member selected from an amorphane, a valencane, a cadinane, an eremophilane, a guaiane, a germacrane and a eudesmane.

17. The method according to claim 15, wherein the sesquiterpene is amorpha-4,11-diene.

18. A method of preparing dihydroartemisinic acid: 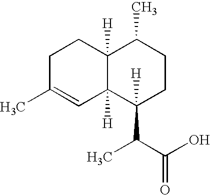
said method comprising:
(a) regioselectively epoxidizing the exocyclic alkene in amorpha-4,11-diene according to the method of claim 9 to form a compound comprising an epoxide moiety and having the formula: 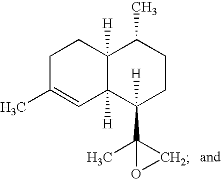
b) hydrolytically opening the epoxide ring to form a diol, thus producing a compound with the formula: 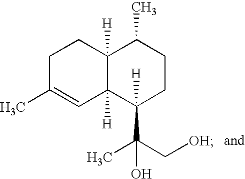
(c) eliminating the tertiary hydroxy group to form an exocyclic alkene, thus producing a compound with the formula: 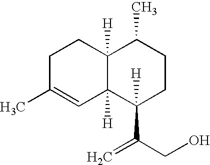
(d) reducing the double bond, thereby preparing a compound of the formula 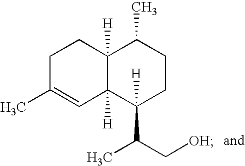
(e) oxidizing the alcohol moiety to a carboxylic acid moiety, thereby preparing dihydroartemisinic acid.

19. A method of preparing dihydroartemisinic acid:
said method comprising:
(a) regioselectively dihydroxylating the exocyclic alkene in amorpha-4,11-diene according to the method of claim 17 to form a diol, thus producing a compound with the formula: 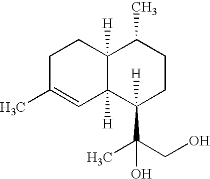
(b) eliminating the tertiary hydroxy group to form an exocyclic alkene, thus producing a compound with the formula: 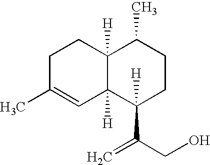
(c) reducing the double bond, thereby preparing a compound of the formula 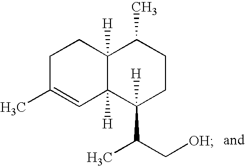
(d) oxidizing the alcohol moiety to a carboxylic acid moiety, thereby preparing dihydroartemisinic acid.

20. A method of preparing dihydroartemisinic acid: 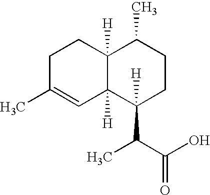
said method comprising:
(a) converting amorpha-4,11-diene: 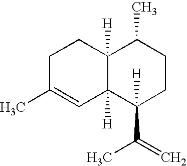
in one step to a compound comprising an alcohol moiety and having the formula: 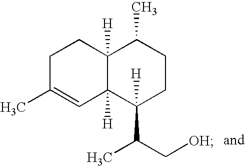
(b) oxidizing the alcohol moiety to a carboxylic acid moiety, thereby preparing the dihydroartemisinic acid.

21. The method according to claim 20, wherein said alcohol is formed by:
(c) regioselectively hydroborating said amorphadiene with a hydroboration reagent capable of reacting selectively with an exocyclic alkene moiety over an endocyclic alkene moiety.

22. The method according to claim 21, wherein said hydroboration reagent is a dicycloalkyl borane.

23. The method according to claim 20, further comprising, prior to step (a), separating said amorphadiene from a mixture comprising a recombinant organism by which said amorphadiene was synthesized.

24. The method according to claim 23, wherein said amorphadiene separated from said mixture is isolated in an amount of at least one kilogram.

25. A method of preparing dihydroartemisinic acid: 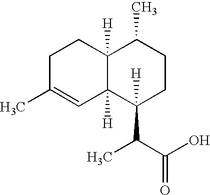
said method comprising:
(a) converting amorpha-4,11-diene: 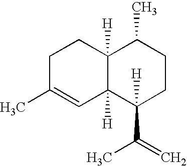
to a compound comprising an alcohol moiety and having the formula: 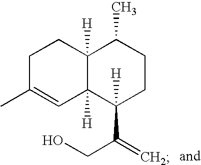
(b) oxidizing the alcohol moiety to a carboxylic acid moiety, thus forming a compound having the formula of artemisinic acid, and
(c) reducing the double bond, thereby preparing the dihydroartemisinic acid.

26. The method according to claim 25, wherein said compound comprising an alcohol moiety is synthesized by:
(i) regioselectively forming an exocyclic allylic anion by reaction of amorphadiene with an alkyl lithium reagent; and
(ii) quenching said exocyclic allylic anion with oxygen, thereby synthesizing said compound comprising an alcohol moiety.

27. The method according to claim 25, wherein said compound comprising an alcohol moiety is synthesized by:
(i) regioselectively forming an exocyclic allylic anion by reaction of amorphadiene with an alkyl lithium reagent;
(ii) reacting said exocyclic allylic anion with an alkyl borate, thus forming a borate ester; and
(iii) oxidizing said borate ester with hydrogen peroxide.

28. The method according to claim 25, wherein the double bond is reduced by subjecting said artemisinic acid to catalytic hydrogenation in the presence of a transition metal catalyst to enatioselectively furnish the dihydroartemisinic acid.

29. The method according to claim 25 wherein step (b) is accomplished by:
(i) oxidizing the allylic alcohol to a compound comprising an aldehyde moiety and having the formula: 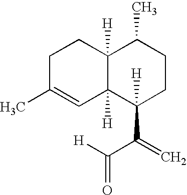
(ii) oxidizing said compound containing an aldehyde moiety to afford the compound having the formula of artemisinic acid.

30. The method according to claim 25, further comprising, prior to step (a), separating said amorphadiene from a mixture comprising a recombinant organism by which said amorphadiene was synthesized.

31. The method according to claim 30, wherein said amorphadiene separated from said mixture is isolated in an amount of at least one kilogram.

32. A method of preparing dihydroartemisinic acid: 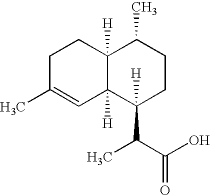
said method comprising:
(a) converting amorphadiene: 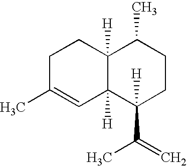
to a compound comprising an alcohol moiety and having the formula: 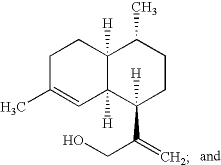
(b) reducing the double bond, thereby preparing a compound of the formula 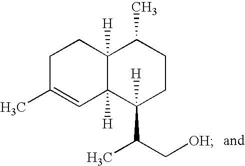
(c) oxidizing the alcohol moiety to a carboxylic acid moiety, thereby preparing the dihydroartemisinic acid.

33. A method according to claim 32 wherein step (b) is accomplished by subjecting said compound comprising an alcohol moiety to catalytic hydrogenation in the presence of a metal catalyst to stereoselectively furnish the reduced alcohol,
wherein said metal catalyst is a member selected from chiral and achiral.

34. A method according to claim 32 wherein step (c) is carried out in two stages, comprising:
(i) oxidizing the saturated alcohol to produce a compound comprising an aldehyde moiety and having the formula: 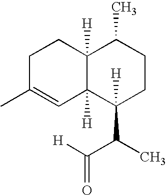
(ii) further oxidizing the compound comprising an aldehyde moiety to produce the dihydroartemisinic acid.

35. The method according to claim 32, further comprising, prior to step (a), separating said amorphadiene from a mixture comprising a recombinant organism by which said amorphadiene was synthesized.

36. The method according to claim 32, wherein said amorphadiene separated from said mixture is isolated in an amount of at least one kilogram.

37. A method of preparing dihydroartemisinic acid: 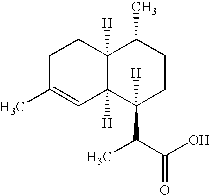
said method comprising:
(a) subjecting amorphadiene: 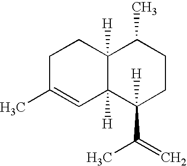
to an “ene” halogenation, thus furnishing a compound having the formula: 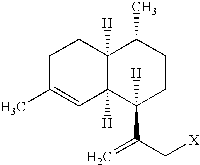
wherein X is a halogen; and
(b) converting the product of step (a) to a compound with the formula: 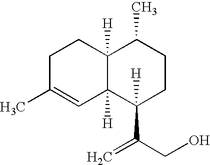
(c) reducing the exocyclic double bond, thereby preparing a compound of the formula 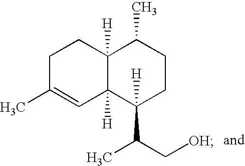
(d) oxidizing the alcohol moiety to a carboxylic acid moiety, thereby preparing the dihydroartemisinic acid.

38. A method of preparing artemisinin: 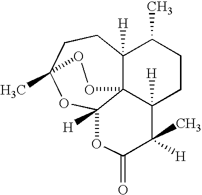
said method comprising:
(a) converting dihydroartemisinic acid or an esterified derivative thereof to an oxidized species using an oxidation procedure, wherein the oxidation procedure is a member selected from photochemical oxidation and non-photochemical oxidation;
(b) subjecting the product of step (a) to an acid or metal catalyzed rearrangement reaction;
(c) oxidizing the product of step (b);
(d) subjecting the product of step (c) to two acid catalyzed cyclizations in order to produce artemisinin.

39. The method of claim 38, wherein said dihydroartemisinic acid is prepared by one of the methods in claim 18, claim 19, claim 20, claim 25, claim 32 and claim 37.

40. The method of claim 38, wherein said photochemical oxidation comprises contacting, with light, a mixture comprising dihydroartemisinic acid, oxygen and a singlet oxygen photosensitizer.

41. The method of claim 40, wherein said photosensitizer is a member selected from methylene blue and rose Bengal.

42. The method according to claim 38, wherein said oxidized species is a hydroperoxide and said hydroperoxide is generated in the presence of a member selected from a peroxide, an endoperoxide and an ozonide.

43. The method of claim 38, wherein said non-photochemical oxidation is accomplished in the presence of hydrogen peroxide and a metal catalyst.

44. The method of claim 43, wherein the metal in the metal catalyst is a member selected from lanthanum, cerium, molybdenum, calcium, tungsten, scandium, titanium, zirconium and vanadium.

45. The method of claim 43, wherein the metal catalyst is supported on a solid inorganic or organic medium which is a member selected from alumina, silica, a zeolite and an organic polymer.

46. The method of claim 43, wherein the metal catalyst is sodium molybdate.

47. The method of claim 38, wherein the metal catalyst of step (b) is a copper salt.

48. The method of claim 47, wherein the copper salt is a member selected from copper (II) trifluoromethanesulfonate, copper (II) sulfate, copper (II) acetate, copper (II) acetylacetonate, and copper (II) chloride.

49. The method of claim 38, wherein the acid in step (d) (acid catalyzed cyclizations) has a pKa of between 5 and −20.

50. The method of claim 38, wherein at least one of said acids in step (d) is a protic acid.

51. The method of claim 50, wherein said protic acid is a member selected from acetic acid, trifluoroacetic acid, methanesulfonic acid, citric acid, p-toluenesulfonic acid and oxalic acid.

52. The method of claim 38, wherein the acid in step (d) is a substance comprising a polymeric backbone or matrix containing acidic functional groups.

53. The method of claim 52, wherein the polymeric backbone or matrix is a member selected from styrene-divinylbenzene compolymer, an acrylate, a methacrylate, a phenol-formaldehyde condensate, an epichlorohydrin amine condensate and a perfluorinated ionomer.

54. The method according to claim 52, wherein the acidic functional groups on the polymeric backbone or matrix are members selected from sulfonates, phosponates and carboxylic acids

55. The method of claim 38, wherein the acid in step (d) is an acidic resin.

56. The method of claim 55, wherein the acidic resin is sulfonated polystyrene.

57. A method of preparing artemisinin: 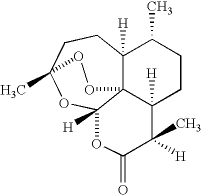
said method comprising:
(a) converting the carboxylic acid moiety on dihydroartemisinic acid to a carboxylic acid derivative moiety, wherein said carboxylic acid derivative moiety is a member selected from esters, acid chlorides, acid bromides, acid anhydrides, amides, thioacids, and thioesters;
(b) subjecting the product of step (d) to an oxidation procedure, wherein the oxidation procedure is a member selected from photochemical oxidation and non-photochemical oxidation;
(c) subjecting the product of step (e) to an acid or metal catalyzed rearrangement reaction;
(d) oxidizing the product of step (f); and
(e) subjecting the product of step (g) to two acid catalyzed cyclizations in order to produce artemisinin.

58. A method of preparing an artemisinin analog, said method comprising:
(a) converting amorphadiene: 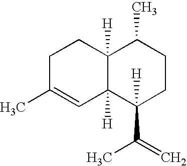
to a compound comprising an alcohol moiety and having the formula: 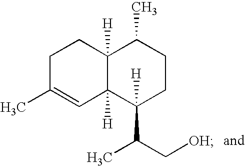
(b) oxidizing the alcohol moiety to an aldehyde moiety, thus producing a dihydroartemisinic aldehyde having a structure according to 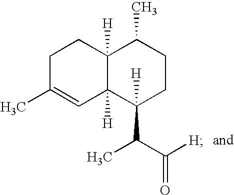
(c) reducing the aldehyde moiety on dihydroartemisinic aldehyde to an alcohol moiety, thereby producing a compound having a structure according to 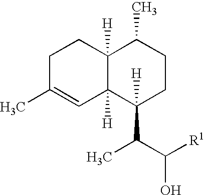
wherein R¹ is a member selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl;
(d) subjecting the product of step (c) to a photooxidative reaction; and
(e) subjecting the product of step (d) to an oxidation-ring closure reaction, thus producing said artemisinin analog, wherein said artemisinin analog has a structure according to [chemical structure]
